(19) United States
(12) Patent Application Publication
(10) Pub. No.: US 2011/0306106 A1
(43) Pub. Date: Dec. 15, 2011

(54) HIGH EXPRESSION PROMOTER AND METHOD FOR PRODUCING GENE PRODUCT USING SAME

(75) Inventors: Jun Sakurai, Tokushima-shi (JP); Masahiro Nagahama, Tokushima (JP); Masataka Oda, Tokushima (JP)

(73) Assignee: OTSUKA CHEMICAL CO., LTD, OSAKA-SHI (JP)

(21) Appl. No.: 13/144,737

(22) PCT Filed: Jan. 15, 2010
(86) PCT No.: PCT/JP2010/050387

§ 371 Date: Aug. 31, 2011

(30) Foreign Application Priority Data
Jan. 16, 2009 (JP) 2009-007950

(51) Int. Cl. C12P 1/04; C12N 1/21; C12N 1/20; C12N 15/75

(52) U.S. Cl. 435/170; 435/320.1; 435/252.31; 435/252.5

(57) ABSTRACT

Disclosed is a promoter which enables the expression of a gene product in a large quantity in Bacillus subtilis. Also disclosed is a method for producing a gene product using the promoter. Specifically disclosed are: a nucleic acid molecule which contains a promoter region derived from a toxin gene of a bacterium belonging to the genus Clostridium and can enhance the expression of a heterologous gene operably linked to the nucleic acid molecule; a nucleic acid construct which contains the nucleic acid molecule and the heterologous gene; a vector carrying the nucleic acid construct; a host cell which is transformed with the vector; and a method and a kit for producing an expression product of the heterologous gene using the nucleic acid molecule, the nucleic acid construct, the vector or the host cell.

### TECHNICAL FIELD

The present invention relates to a high-expression promoter and a process for producing a gene product using said promoter. Specifically, the invention relates to a nucleic acid molecule comprising a promoter region derived from a toxin gene of Clostridium bacterium, a nucleic acid construct comprising said nucleic acid molecule and a heterologous gene operably linked thereto, a vector comprising them, a host cell transformed with said vector, and a process and a kit for the production of a gene product using the same.

### BACKGROUND ARTS

Progresses in the genetic engineering have widely enabled us to introduce and express a desired gene in a microorganism such as bacteria and yeasts and a cultured cell of a higher animal such as a mammal, to produce various gene products such as proteins. Prevailing among such production method is a procedure using Escherichia coli (E. coli), for their ease in culturing operation, high reproductivity and high level of gene expression, and such procedure is widely used in industry. However, a gene product that has been expressed in a Gram-negative bacterium E. coli is accumulated in the periplasm between the inner and outer membranes. Therefore, in order to extract the gene product it is necessary to first destroy the cell body before separating and purifying the product of interest. Further, since LPSs (lipopolysaccharides), which exist on the outer membrane of E. coli, act as pyrogens in a living organism such as human, it is necessary to remove such pyrogens when a gene product produced employing E. coli is to be used, requiring a considerable time and cost for the purification of the gene product.

A Gram-positive bacterium Bacillus subtilis (B. subtilis) has been drawing attention as a bacterial species having no such disadvantages. B. subtilis does not possess a pyrogen as above, does not have a pathogenicity, and in many cases secretes its gene product to the outside of the cell body. It is therefore useful for the production of a gene product which is to be applied to a living organism as, for example, a medicament or food. However, B. subtilis has a disadvantage that the level of its production of a gene product is relatively low compared to E. coli, which hinders the industrial use of this bacterial species. One reason for this low-level production of a gene product by B. subtilis is the absence of a promoter with good operability and expression efficacy like lac promoter of E. coli. Given such circumstances, there have been attempts to search for a promoter that effectively operates in B. subtilis.

For instance, Patent Literature 1 describes a promoter containing a particular sequence; Patent Literature 2 describes a hybrid promoter containing a promoter of the alpha-amylase gene of Bacillus amyloliquefaciens and an enhancer receptor such as those originate from the alkaline protease gene of B. subtilis; Patent Literature 3 describes a DNA fragment which comprises an upstream region of alkaline cellulase K-64 and which may increase the expression of structural genes attached to the downstream thereof; Patent Literature 4 describes a promoter which is derived from a variant of the alpha-amylase gene promoter of Bacillus licheniformis; Patent Literature 5 describes promoters comprising an altered AmyQ promoter sequence, cryIIIA mRNA stabilizing sequence and a sequence in which these sequences are arranged in tandem; Patent Literature 6 describes an altered promoter in which a special sequence has been introduced near the 3′ terminal of the alpha-amylase gene promoter of Bacillus amyloliquefaciens; Patent Literature 7 describes promoters derived from the upstream regions of several genes which are expressed in a stationary phase-specific manner in B. subtilis; and Non-Patent Literature 1 describes a promoter derived from the MWP gene of Bacillus brevis.

However, none of the above-mentioned promoters has yielded a satisfactory result, leaving a need for further development of promoters.

### PRIOR ART LITERATURES

### Patent Literatures

Patent Literature 1: JPA 60-137291
Patent Literature 2: JP A 6-500689
Patent Literature 3: JPA 6-217781
Patent Literature 4; JP A 7-504085
Patent Literature 5: JP A 2002-504379
Patent Literature 6; JP A 2002-272466
Patent Literature 7: WO 2002/072819

### Non-Patent Literatures

Non-Patent Literature 1: Yamagata et al., Proc Natl Acad Sci USA. 1989 May; 86(10):3589⁻⁹³

### SUMMARY OF THE INVENTION

### Problems to be Solved by the Invention

An object of the present invention is to provide a promoter that is capable of a high level expression of a gene product in B. subtilis, and a process for the production of a gene product utilizing said promoter.

### Means for Solving the Problems

The inventors carried on an intensive research in order to solve the problems above, and found that the expression of a gene product was dramatically increased by linking an upstream sequence of a toxin gene of Clostridium bacterium, especially the iota (O-toxin of Clostridium perfringens (C. perfringens), to a heterologous gene and expressing it in B. subtilis, thereby completing the invention. Namely, the invention relates to the followings:

(1) A nucleic acid molecule comprising a promoter region derived from a toxin gene of a Clostridium bacterium, which enhances the expression of a heterologous gene operably linked thereto.
(2) The nucleic acid molecule of (1), wherein the toxin gene is selected from the group consisting of C. perfringens alpha (α)-toxin, epsilon (ε)-toxin and iota (ι)-toxin, botulinum toxin, and tetanus toxin.
(3) A nucleic acid construct comprising the nucleic acid molecule of (1) or (2) above and a heterologous gene operably linked thereto.
(4) A vector comprising the nucleic acid molecule of (1) or (2) above or the nucleic acid construct of (3) above.
(5) A host cell that has been transformed with the vector of (4) above.
(6) A process for the production of a gene product, comprising the step of culturing the host cell of (5) above.
(7) A kit for the production of a gene product, comprising the nucleic acid molecule of (1) or (2) above, the nucleic acid construct of (3) above, the vector of (4) above, and/or the host cell of (5) above.

### The Effects of the Invention

By the present invention, the levels of expression of various gene products can be remarkably increased in B. subtilis, which had been considered to be difficult to be used for a high-level expression of a gene product. Therefore, the invention allows obtaining a large amount of a pyrogen-free, biologically-safe gene product by a simple purification operation, being expected to provide a great contribution in the fields of medical and food products, in particular.

In addition, the kit for the production of a gene product of the present invention is capable of producing various types of gene product readily and in large amount, and therefore can advantageously be utilized in a research institute where many types of small lots of gene product are required to be expressed.

### BRIEF DESCRIPTION OF THE DRAWINGS

FIG. 1 is a schematic diagram showing the location of C. perfringens ι-toxin promoter (black arrow) in pCIP.

FIG. 2 is a diagram showing the sequence of the region of pCIP where C. perfringens ι-toxin promoter has been inserted. In the figure, the shaded bold part shows the inserted promoter region and the underlined parts show the restriction sites, respectively.

### DESCRIPTION OF EMBODIMENTS

Unless otherwise stated herein, the scientific and technical terms used in the context of the present invention have meanings which are normally understood by those having an ordinary skill in the art. In general, the terms and the techniques used in the context of the cell and cell culture, molecular biology, microbiology, genetics, protein and nucleic acid chemistry, and hybridization described herein are those which are well known and normally used in the art. Unless specifically stated, the methods and the techniques of the present invention are performed according to the routine methods well known in the art, as described in various references cited herein. Such references include, for example, Sambrook et al., Molecular Cloning: A Laboratory Manual, 2d ed., Cold Spring Harbor Laboratory Press (1989) and Sambrook et al., Molecular Cloning: A Laboratory Manual, 3d ed., Cold Spring Harbor Press (2001), Ausubel et al., Current Protocols in Molecular Biology, Greene Publishing Associates (1992, and 2000 suppl.), Ausubel et al., Short Protocols in Molecular Biology: A Compendium of Methods from Current Protocols in Molecular Biology—4th Ed., Wiley & Sons (1999), Harlow and Lane, Antibodies: A Laboratory Manual, Cold Spring Harbor Laboratory Press (1990), and Harlow and Lane, Using Antibodies: A Laboratory Manual, Cold Spring Harbor Laboratory Press (1999).

The enzymatic reactions and purification techniques are to be performed in accordance with the instruction of the manufacturer, as normally carried out in the art or as described herein. The terms, experimental procedures and techniques used in the context of the analytical chemistry and synthetic organic chemistry described herein are those which are well known and normally used in the art. Also, the standard techniques in the art are to be used in genetic engineering, cell culturing, chemical synthesis and chemical analysis, etc.

An aspect of the present invention relates to a nucleic acid molecule comprising a promoter region derived from a toxin gene of a Clostridium bacterium, which enhances the expression of a heterologous gene operably linked thereto.

The Clostridium bacterium in the present invention is not particularly limited as long as it has a toxin gene, and includes, for example, Clostridium perfringens (C. perfringens), Clostridium botulinum (C. botulinum), Clostridium tetani (C tetaa, C. novyi, C. septicum, C. histolycum, C. difficile, etc.

The toxin gene of the Clostridium bacterium in the present invention is not particularly limited as long as it comprises in its upstream region a sequence having a promoter activity, and includes, for example, C. perfringens α-toxin gene (see, e.g., Titball et al., Infect Immun. 1989 February; 57(2)367-76), ε-toxin gene (see, e.g., Hunter et al., Infect Immun. 1992 January; 60(1): 102-10) and ι-toxin gene (see, e.g., Perelle et al., Infect Immun. 1993 December; 61(12): 5147-56), botulinum toxin gene (see, e.g., Whelan et al., Eur J. Biochem. 1992 Mar. 1; 204(2): 657-67, Thompson et al., Eur J. Biochem. 1990 Apr. 20; 189(1): 73-81), tetanus toxin gene (see, e.g., Eisel et al., EMBO J. 1986 October; 5(10): 2495-502), etc. The nucleic acid sequences of these genes are known and available from the references above and the genetic databases such as NCBI. For instance, the gene sequences of C. perfringens α-toxin, ε-toxin and ι-toxin have been registered at and freely available from GenBank by Accession Nos. X13608, M80837 and X73562; the gene sequences of types A to F botulinum toxins by X52066, X78229, S74768, S49407, X62683 and X71086; and the gene sequence of the tetanus toxin by X04436, respectively.

In the present invention, a promoter region means a gene region present in the upstream of a coding region (structural gene) of a gene, e.g., in 5′ non-coding region, and whose presence allows initiating the transcription of the structural gene. A promoter region typically contains a −10 region (also known as pribnow box) which is located at about 10 bases upstream (−10) to the transcription initiation site (+1), and has a motif such as 5′-TATAAT-3′ or its analogous sequence and/or a −35 region located at about 35 bases upstream (−35) and having a motif such as 5′-TTGACA-3′ or its analogous sequence. Thus, in one embodiment of the present invention, a promoter region includes a 5′ non-coding region of a gene selected from the group consisting of C perfringensa-toxin gene, ε-toxin gene and ι-toxin gene, botulinum types A and E toxin genes and tetanus toxin gene, more typically, a sequence according to SEQ ID NOs: 1 to 6 or a partial sequence thereof.

Preferred partial sequence is, or comprises, a −35 region and/or a −10 region, namely, positions 681 to 709 of SEQ ID NO: 1 (SEQ ID NO: 7), positions 97 to 125 of SEQ ID NO: 2 (SEQ ID NO: 8), positions 85 to 114 of SEQ ID NO: 3 (SEQ ID NO: 9), positions 19 to 46 of SEQ ID NO: 4 (SEQ ID NO: 10), positions 103 to 108 of SEQ ID NO: 5 (SEQ ID NO: 11) or positions 193 to 200 of SEQ ID NO: 6 (SEQ ID NO: 12). A sequence comprising a −35 region and/or −10 region includes for example, without limitation, a portion from a −35 region or −10 region to the start codon, which is, for each of the above-mentioned toxin gene, specifically, a sequence of SEQ ID NOs: 13 to 18. The portion to be selected as the partial sequence may be appropriately determined in consideration of various conditions such as the level of the promoter activity or the presence of an appropriate restriction site. The level of the promoter activity may be assessed, for example, by comparing the levels of gene expressions which are operably linked to the subject partial sequence, as described below. Also, a restriction site on a nucleic acid sequence can readily be determined using tools for genetic analysis available on Internet, such as Webcutter (http://www.firstmarket.com/cutter/cut2.html) or NEBcutter (http://tools.neb.com/NEBcutter2/index.php). In case where no desired restriction site is present, a restriction site may be added into the partial sequence by site-specific mutagenesis, etc., as described hereinbelow.

On the other hand, a nucleic acid molecule which does not have a typical motif as above, but still exists in the upstream of the structural gene of a Clostridium toxin gene and is capable of initiating the transcription of the gene is also encompassed in the promoter of the present invention. A promoter region may be identified based on the presence of a typical motif as above; or all or a part of the 5′ non-coding region of a gene may conveniently be taken as a promoter region. Since it appears that RNA polymerase, which is involved in mRNA synthesis, binds to a promoter region, it is also possible to identify a promoter region based on the sequence recognized by RNA polymerase. The part of the gene which would actually function as a promoter may be identified, for example, by mutating (e.g., partially deleting or substituting) the upstream region of the coding region and assessing the change in the gene expression. When the gene expression is decreased by the mutation, the mutated part is the promoter region, or includes the promoter region, or partially overlaps with the promoter region.

The nucleic acid molecule of the present invention may also comprise a nucleic acid molecule having a nucleic acid sequence analogous to the promoter region of a naturally occurring Clostridium toxin gene and having an activity equivalent to that of said region, e.g., a transcription promoting activity (a promoter activity). The above-mentioned “analogous nucleic acid sequence” includes, for example, a nucleic acid sequence having 60% or greater, 65% or greater, 70% or greater, 75% or greater, 80% or greater, 85% or greater, 90% or greater, 95% or greater, 98% or greater, or 99% or greater homology to the nucleic acid sequence of a naturally occurring promoter region (e.g., a nucleic acid sequence according to SEQ ID NOs: 1 to 6); or a nucleic acid sequence of a nucleic acid molecule which hybridizes to a naturally occurring promoter region (e.g., a nucleic acid molecule encoded by a nucleic acid sequence according to SEQ ID NOs: 1 to 6) under stringent conditions.

Herein, a homology is a term well known in the art, and refers, for example, to the proportion of bases that match between multiple nucleic acid sequences when they are appropriately aligned. A homology of nucleic acid sequences may be determined utilizing tools publicly available on Internet, e.g., BLAST (http://blast.ncbi.nlm.nih.gov/Blast.cgi) or various commercial software.

The term “stringent condition” used herein is a well known parameter in the art and described in standard protocols such as Sambrook et al., Molecular Cloning: A Laboratory Manual, 3d ed., Cold Spring Harbor Press (2001) and Ausubel et al., Current Protocols in Molecular Biology, Greene Publishing Associates (1992).

A stringent condition in the present invention refers to a hybridization at 65° C. in a hybridization buffer comprised of 3.5×SSC (0.15M sodium chloride/0.15M sodium citrate, pH 7), Ficoll 0.02%, polyvinylpyrrolidone 0.02%, bovine serum albumin 0.02%, NaH₂PO₄ 25 mM (pH 7), SDS 0.05% and EDTA 2 mM. After hybridization, the membrane to which DNA was transferred is washed in 2×SSC at room temperature, then in 0.1 to 0.5×SSC/0.1×SDS at a temperature up to 68° C. Alternatively, a stringent hybridization may be performed using a commercially available hybridization buffer such as ExpressHyb™ Hybridization Solution (Clontech), under the conditions for hybridization and washing described by the manufacturer.

There will be other conditions and reagents which may be used to give a similar level of stringency, though these are not described in particular herein since a person with an ordinary skill in the art should be familiar with such conditions. However, a condition may be appropriately adjusted in order to allow an unambiguous identification of a sequence analogous to a promoter region of a Clostridium toxin gene.

A promoter region in the present invention may be one included in a naturally occurring Clostridium toxin gene or may be a variant of a naturally occurring promoter region with a mutation. A mutation includes a deletion, substitution or addition of any nucleotide sequence, e.g., an alteration other than in the −10 region and/or the −35 region, an alteration in a part from the −10 region to the −35 region (including a part between both regions), an alteration of a motif in the −10 region and/or the −35 region, an increase/decrease in the number of nucleotide and/or substitution of nucleotides between these regions, and an increase/decrease in the number of nucleotide and/or substitution of nucleotides between either of these regions and the transcription initiation site.

In one embodiment of the present invention, a preferred variant of a nucleic acid molecule is a nucleic acid molecule which comprises one or more mutations in the nucleic acid sequence of SEQ ID NO: 1 and comprises the nucleic acid sequence of SEQ ID NO: 7; a nucleic acid molecule which comprises one or more mutations in the nucleic acid sequence of SEQ ID NO: 2 and comprises the nucleic acid sequence of SEQ ID NO: 8; a nucleic acid molecule which comprises one or more mutations in the nucleic acid sequence of SEQ ID NO: 3 and comprises the nucleic acid sequence of SEQ ID NO: 9; a nucleic acid molecule which comprises one or more mutations in the nucleic acid sequence of SEQ ID NO: 4 and comprises the nucleic acid sequence of SEQ ID NO: 10; a nucleic acid molecule which comprises one or more mutations in the nucleic acid sequence of SEQ ID NO: 5 and comprises the nucleic acid sequence of SEQ ID NO: 11; or a nucleic acid molecule which comprises one or more mutations in the nucleic acid sequence of SEQ ID NO: 6 and comprises the nucleic acid sequence of SEQ ID NO: 12.

The number of the nucleotide to be mutated is not particularly limited, though it may be in the range of one to several, 1 to 10, 1 to 20, 1 to 30, 1 to 50, or 1 to 100.

Among the variants described above, a mutation that elevates the promoter activity, namely, a mutation that increases the expression of a gene of the interest, is preferred. Moreover, a desired restriction site may be added to a promoter region in the present invention for the convenience in cloning. The method for introducing a restriction site into a promoter region is not particularly limited and may be performed using any known procedures such as, for example, various methods for site-specific mutagenesis such as the inverse PCR, overlap extension PCR and megaprimer PCR, and a method of amplification, e.g., by PCR, using a primer to which a restriction site has been added.

The nucleic acid molecule of the present invention is typically a DNA, although it may be, in some cases, an RNA or an artificial nucleic acid such as PNA, LNA or various modified nucleic acids (such as phosphorothioate modified nucleic acid).

A heterologous gene in the present invention means a gene which is different from a gene from which the promoter region included in the nucleic acid molecule of the present invention has been derived. A species of the organism may be the same or different. For instance, if the nucleic acid molecule of the present invention derives from C. perfringens ι-toxin gene, a heterologous gene may be any gene other than C. perfringens ι-toxin gene, e.g., without limitation, C. perfringens α-toxin gene, s-toxin gene, botulinum toxin gene or tetanus toxin gene.

A preferred heterologous gene in the present invention includes, without limitation, e.g., a gene derived from bacteria, preferably Gram-positive bacteria (such as C. perfringens α-toxin, s-toxin and t-toxin, botulinum toxin and tetanus toxin), a gene of a low molecular weight protein (e.g., those of a molecular weight at or less than 100 kDa, especially a molecular weight at or less than 50 kDa), a gene of a protein with a drug action (such as an enzyme, hormone, antibody and cytokine originates from the subject to be treated, such as a human or non-human animal). A heterologous gene of the present invention typically encodes for a protein, thought it may encode for a nucleic acid molecule such as an antisense nucleic acid or an siRNA.

A heterologous gene may comprise only a coding region, or may comprise a non-coding region. A non-coding region may also comprise various functional sequences such as a promoter, an enhancer, a terminator or a ribosome-binding sequence.

A heterologous gene may also comprise various alterations that are advantageous for the expression, secretion and/or purification of a gene product. Such alteration includes, without limitation, e.g., an addition of a secretory signal sequence, an addition of a tag sequence for purification (such as His tag, GST tag, S tag, T7 tag).

A secretory signal includes, without limitation, e.g., those derived from acetolactate decarboxylase, alkaline cellulase, alkaline phosphatase, alkaline protease, amylase (such as α-amylase), bacillopeptidase, chitinase, cyclodextrin glucanotransferase, β-glucanase, β-lactamase, levanase, levansucrase, β-mannanase, metalloprotease, MWP (middle wall protein), neutral protease, OWP (outer wall protein), RNase, sphingomyelinase, subtilisin and xylanase of a Bacillus bacterium (for specific sequence, see, e.g., Microbiol Rev. 1993 March; 57 (1)109-37).

In the present invention, by a heterologous gene being “operably linked” to a nucleic acid molecule of the present invention, it is meant that the heterologous gene and the nucleic acid molecule of the present invention are chemically bound such that, when a nucleic acid construct of the present invention comprising the nucleic acid molecule of the present invention and the heterologous gene operably linked thereto is introduced into an appropriate host cell, said heterologous gene will be expressed in a normal condition in that host cell. A normal condition refers to, in a host cell, a state in which there has been no treatment which would enhance or decrease the expression of said heterologous gene, or a state in which the cell is not suffering from a pathogen which would enhance or decrease the expression of said heterologous gene. As long as the above condition is satisfied, a nucleic acid molecule of the present invention may be linked to either the upstream or downstream of the heterologous gene, though it typically is located to the upstream of the heterologous gene. Here, the upstream and downstream of a heterologous gene means the 5′ side and 3′ side of the heterologous gene, respectively. The nucleic acid molecule of the present invention may be directly linked to a heterologous gene, or may be linked via an intervening nucleotide sequence (a spacer). The positional relationship between a nucleic acid molecule of the present invention and a heterologous gene is preferably identical to or similar to the positional relationship of a promoter region contained in the nucleic acid molecule of the present invention and the coding region of the gene from which the promoter is derived from, though it is not limited as long as the conditions above are fulfilled.

In the present invention, by “linking” a nucleic acid molecule of the present invention and a heterologous gene, it is meant that the nucleic acid molecule of the present invention and the heterologous gene are chemically bound either directly or via a spacer. In the present invention, when the nucleic acid molecule is a DNA or an RNA, such a chemical binding typically includes a phosphodiester linkage, though it may be other linkage such as phosphorothioate linkage as long as it is operable.

In the present invention, by a nucleic acid molecule of the present invention will “enhance the expression” of a heterologous gene, it is meant that, by operably linking the heterologous gene to the nucleic acid molecule of the present invention, the expression of the heterologous gene in an appropriate host cell will be elevated compared to when it is not linked to the nucleic acid molecule of the present invention. The elevation of the expression may be assessed as an increase in the expression level per unit time, and/or as a shortening of the period to obtain a given amount of expression product. In the present invention, a level of expression is higher than when the nucleic acid molecule of the present invention is not linked, preferably 1.5-fold or higher, more preferably 2-fold or higher, further preferably 2.5-fold or higher, yet more preferably 3-fold or higher, particularly preferably 4-fold or higher. The enhancing effect of the nucleic acid molecule of the present invention on the expression of a heterologous gene may vary depending on the type of the heterologous gene to be linked.

The amount of the expression product may be assessed by culturing for a given time period a host cell in which the nucleic acid construct of the present invention has been introduced, and measuring the amount of the expression product within the culture supernatant or within the host cell lysate using known procedures. Such procedures include, without limitation, e.g., general methods for protein measurement including methods using antibodies such as EIA, ELISA, IRA, IRMA and Western blotting, ultraviolet absorption method, colorimetric methods such as Bradford method (Coomassie blue method), Lowry method (phenol reagent method), BCA method (bicinchoninic acid method), quantification by the comparison of electrophoresis patterns, as well as various other procedures based on the nature of the expression product of the heterologous gene including, e.g., when the expression product is an enzyme, quantification of the reaction with its substrate, or, when the expression product is a substrate of an enzyme, quantification of the reaction with its enzyme, or, when the expression product is a physiologically active substance, quantification of the physiological reaction.

A level of expression may also readily be compared using as a heterologous gene a reporter gene which is easy to be detected, such as CAT (chloramphenicol acetyltransferase), DsRed (Discosoma sp. Red Fluorescent Protein), Green Fluorescent Protein (GFP), β-glucuronidase (GUS), lacZ and luciferase.

The nucleic acid molecule of the present invention may comprise two or more promoter regions. In this case, at least one of the promoter regions is that derived from Clostridium toxin gene. Accordingly, the nucleic acid molecule of the present invention may comprise at least one promoter region which is derived from Clostridium toxin gene, and at least one promoter region which is not derived from Clostridium toxin gene. Two or more promoter regions may be linked to each other either directly or via intervening sequence.

The nucleic acid molecule of the present invention may also comprise a ribosome binding site such as Shine-Dalgarno (SD) sequence which is involved in translation. SD sequence is a purine base-rich, 3 to 9-base-long sequence having a motif such as 5′-AGGAGG-3′ or its analogous sequence, and is considered to be bound by 16SrRNA. A ribosome binding site is preferably contained in downstream of the transcription initiation site. A nucleic acid molecule of the present invention comprising SD sequence includes, for example, those comprising the sequences of SEQ ID NOs: 1 to 6, or the partial sequences thereof, i.e., the sequences described in SEQ ID NOs: 19 to 24.

The present invention also relates to a nucleic acid construct comprising a nucleic acid molecule of the present invention and a heterologous gene operably linked thereto. The nucleic acid construct of the present invention may have, besides the nucleic acid molecule of the present invention and the heterologous gene, a terminator that terminates the transcription (also referred to as a transcription termination signal). A terminator may be a part of the heterologous gene, or may be derived therefrom, or may not be derived therefrom. General examples of a terminator are well known to those skilled in the art, and any of these may be used. Specifically, it includes, without limitation, e.g., a sequence having a palindromic structure which renders the transcribed mRNA to have a strong hairpin structure (e.g., a GC-rich sequence). The nucleic acid construct of the present invention may also include, besides those aforementioned, other gene sequences which allow an efficient replication and expression of the construct within desired cell.

The nucleic acid construct of the present invention may be introduced into a host cell by various nucleic acid introducing methods, such as calcium phosphate method, lipofection method, ultrasound transfection method, electroporation method, particle gun method, microinjection method, liposome method (e.g., by cationic liposome), competent cell method, protoplast method, to allow the expression of its carrying heterologous gene. Furthermore, the nucleic acid construct of the present invention may be incorporated into various expression vectors, which is then used to transfect a host cell to allow the expression of its carrying heterologous gene.

The present invention also relates to a vector comprising a nucleic acid molecule or a nucleic acid construct of the present invention. In the present invention, a vector means any nucleic acid molecule that is capable of incorporating a nucleic acid molecule or nucleic acid construct of the present invention into itself, maintaining it, and introducing it into a host cell gene, amplifying and/or expressing a heterologous gene. A vector typically is constructed by a DNA, though a vector constructed by an RNA may also be used. A vector includes, without limitation, e.g., a plasmid, cosmid, phage, virus, YAC and BAC. Although a vector may be categorized as, according to its use, such as a cloning vector which is to be used for gene cloning, or as an expression vector to be used for gene expression, the vector of the present invention encompasses vectors of these various uses. A vector may include, according to its use, besides the nucleic acid molecule or nucleic acid construct of the present invention, various functional nucleotide sequences useful for, e.g., the incorporation of the nucleic acid molecule or nucleic acid construct, for introduction into a host cell, for replication and for heterologous gene expression. Such nucleotide sequences include, for example, without limitation, a restriction site (such as multicloning site), a replication origin sequence, a selection marker gene sequence, a reporter gene sequence, a promoter sequence and a terminator sequence. Such functional nucleotide sequences are well known to those skilled in the art, and any of the known functional sequences may be used in an appropriate location and orientation.

A vector of the present invention comprises at least a nucleic acid molecule of the present invention. One embodiment of the vector of the present invention therefore does not comprise any subject heterologous gene. Here, a subject heterologous gene does not comprise any functional gene that render an additional function to the vector, e.g., a selection marker gene or a reporter gene. Accordingly, the above-mentioned embodiment of the vector of the invention does not comprise any subject heterologous gene, though it may comprise such functional genes. In this embodiment, the vector preferably has a restriction site, more preferably multicloning site, which is capable of operably linking the nucleic acid molecule of the present invention and the subject heterologous gene, so that any desired heterologous gene can be incorporated therein. Accordingly, a preferred vector of this embodiment may be used for the expression of any heterologous gene. A particularly preferred example of this embodiment includes, without limitation, a vector having the nucleic acid sequence of SEQ ID NO: 33.

In another embodiment, the vector of the present invention comprises a nucleic acid molecule of the present invention and a heterologous gene operably linked thereto, i.e., a nucleic acid construct of the present invention. A particularly preferred example of this embodiment includes, without limitation, a vector having the nucleic acid sequence of any one of SEQ ID NOs: 30, 58 to 62.

A vector may be generated by combining desired functional nucleotide sequences, or may be generated based on various commercially available vectors. Vectors corresponding to various host cells and for various usages are available. Vectors for Bacillus subtilis including, for example, pA-spac, pAM1, pAX01, pBS72, pC194, pDG1661, pDG1662, pDG1663, pDG1664, pDG1728, pDG1729, pDG1731, pDG271, pDL, pDK, pDH32, pE194, pE194-cop6, pGlt-Cm, pGlt-Kan, pHCM02, pHCM04, pHCM05, pHY500, pHY700, pHY4831, pHT110R2L5, pHT210, pHV1431, pHV1432, pIP404, pIP501, pLS20, pLS32, pMLK83, pMTLBS72, pNDH33, pNH200, pNH300, pNH400, pNU100, pNU200, pNU211, pNU211R2L5, pPyr-Cm, pPyr-Kan, pSac-Cm, pSac-Kan, pSM19035, pT127, pT181, pTA1015, pTA1060, pTB19, pTRKH2, pUB110, φ105 and SPβ; vectors for E. coli including, for example, pACY177, pACYC184, pBR322, pBluesript, pET, pUC18, pUC19, λgt10 and λgt11; vectors which can be used for both B. subtilis and E. coli (such as a shuttle vector) including, for example, pBE20, pBE60, pE18, pEB10, pHB201, pHP13, pHPS9, pHV14, pHY300PLK, pLB5, pRB373, pUB18, pUB19 and pWB980 are known (see, e.g., Schumann, Adv Appl Microbiol. 2007; 62:137-89). Accordingly, one embodiment of the present invention is a vector in which a nucleic acid molecule or nucleic acid construct of the present invention has been incorporated into one of such vector or its modified version.

The incorporation of a nucleic acid molecule or nucleic acid construct of the present invention into a vector is typically carried out with a restriction enzyme. As a restriction enzyme, any known enzyme known to those skilled in the art such as AatII, AccI, ApaI, AorI, BamHI, BglII, BsaAI, BsmI, BssHII, BstXI, Cfr9I, ClaI, DdcI, DpnI, DraI, EcoRI, EcoRV, EcoT22I, FokI, FspI, HaeIII, HapI, HapII, HincII, Hinfl, HindIII, KpnI, MaeIII, MboI, MluI, MseI, MvaI, NaeI, NcoI, NdeI, NheI, NotI, PmaCI, PstI, PvuII, RsaI, SacI, SacII, SalI, Sau3AI, Sau96I, ScaI, SfiI, SmaI, SpeI, SphI, SplI, SspI, TaqI, XbaI, XmnI and XhoI may be used.

The present invention also relates to a host cell transformed with a nucleic acid molecule, nucleic acid construct or vector of the present invention. The host cell in the present invention is not particularly limited as long as a nucleic acid molecule of the present invention can function within that host cell, namely, a heterologous gene that is operably linked to the nucleic acid molecule of the present invention can be expressed within it, and/or, a vector of the present invention can be maintained or replicated within it, and encompasses various cells including prokaryotic cells and eukaryotic cells. In one embodiment of the present invention, a host cell is a prokaryotic cell, typically a bacterial cell. In the present invention, a preferred bacterial species includes without limitation, e.g., a Bacillus bacterium such as Bacillus subtilis (B. subtilis), Bacillus brevis, Bacillus licheniformis, Bacillus megaterium, Bacillus stearothermophilus, and E. coli. Among these, a Bacillus bacterium is preferred for the ease of purifying the expression product and a low risk of contamination of biologically adverse by-product derived from the production steps. Bacillus subtilis is particularly preferred. As for Bacillus subtilis, various bacterial strains, e.g., BD170 strain, 168 strain, ISW1214 strain, MI114 strain are available. Also, a bacterial strain in which the production of extracellularly secreted protease has been suppressed (such as e.g., 106HL strain, DY-16 strain) may be used.

Transforming a host cell typically refers to introducing an exogenous nucleic acid molecule into a host cell to change the genetic characteristics of the cell, which is carried out, specifically, by introducing a nucleic acid molecule, nucleic acid construct or vector of the present invention into a host cell. Herein, such introduced host cell may especially be referred to as a transformant. For introduction, various methods for introducing a nucleic acid may be used, such as calcium phosphate method, lipofection method, ultrasound transfection method, electroporation method, particle gun method, microinjection method, liposome method (e.g., by a cationic liposome), competent cell method and protoplast method. In the case of a nucleic acid molecule or nucleic acid construct, introduction methods utilizing a virus vector such as, e.g., an adenovirus vector or a retrovirus vector may also be used besides the above-mentioned methods. Introduced nucleic acid molecule, nucleic acid construct or vector will be incorporated into the genome of the host cell, which may allow the expression of the subject heterologous gene, either constitutively or inducibly. When a nucleic acid molecule is to be introduced, it is necessary to be incorporated into the host cell genome such that it will be operably linked to the subjected heterologous gene. Moreover, when a vector is to be introduced, the heterologous gene may be expressed within the host cell without being incorporated into the host cell genome.

A host cell may also be used for maintaining or amplifying a vector of the present invention. For such use, the vector is maintained within the host cell, being either independent of or incorporated into the host cell genome, or is autonomously amplified. In this case, the expression of the heterologous gene may or may not be taken place. In another embodiment, the vector is maintained without being replicated within the same host cell, but it will be replicated upon the division of the host cell. The vector thus maintained or amplified may then be introduced into a host cell for expression.

The present invention also relates to a process for the production of a gene product, comprising a step of culturing a host cell of the present invention (i.e., a transformant). A transformant of the present invention may be cultured by known procedures suitable for each transformant. Culturing is carried out typically in a medium. A medium includes those in various forms such as a solid, semi-solid or liquid, though a liquid medium is preferred for the ease of treating and capability of large-scale culture. Furthermore, a medium to be used may be either a natural or artificial medium, as long as it contains a carbon source, a nitrogen source, inorganic salts, etc. which will be assimilated by the transformant and is capable of an efficient culture. A carbon source may be any one of those which will be assimilated by the transformant, and glucose, fructose, sucrose, maltose, cellobiose, a syrup containing them, a carbohydrate such as starch or starch hydrolysate, an organic acid such as acetic acid or propionic acid, alcohols such as ethanol or propanol, etc. may be used. As a nitrogen source, ammonium, ammonium chloride, an ammonium salt of inorganic or organic acid, such as ammonium sulfate, ammonium acetate, ammonium phosphate, and other nitrogen-containing compounds, as well as a peptone, meat extract, fish extract, yeast extract, corn steep liquor, casein hydrolysate, soymeal and soymeal hydrolysate, various fermentative bacteria and their digested materials, etc., may be used. As inorganic salts, potassium dihydrogen phosphate, dipotassium hydrogenphosphate, magnesium phosphate, magnesium sulfate, sodium chloride, ferrous sulphate, manganese sulfate, copper sulfate, calcium carbonate, etc. may be used.

Culturing of a transformant which utilizes as a host a prokaryote such as B. subtilis or E. coli or an eukaryote such as an yeast is carried out under an aerobic condition such as in a normal shaking culture or in a deep aerated and agitated culture. The culturing temperature is typically 15 to 50° C., preferably 20 to 40° C., and the culturing period is normally 16 hours to 7 days. The pH in the culture is maintained from 3.0 to 9.0. The adjustment of pH is done using such as an inorganic or organic acid, alkaline solution, urea, calcium carbonate, and ammonium. Moreover, an antibiotic such as ampicillin and tetracycline may be added into the medium as necessary during the culture. When a transformant of the present invention comprises an inducible promoter, an inducer may be added into the medium as necessary. For instance, when lac promoter is contained, those such as isopropyl-β-D-thiogalactopyranoside, when trp promoter is contained, those such as an indoleacrylic acid may be added into the medium.

A transformant in culture is capable of expressing the subject heterologous gene with or without the induction, and accumulating the product in the culture. The expression product of a heterologous gene may be accumulated within the host cell, secreted outside the host cell, or accumulated on the outer membrane of the host cell, and the manner of the production may be altered by changing the host cell to be used or the structure of the expression product. In the present invention, a manner by which the expression product is secreted outside the host cell is preferred, because the purification of the expression product is easy.

The expression product produced by a transformant of the present invention may be purified as follows. For instance, in case when the expression product is expressed in the cell in dissolved form, after being cultured, the cells are collected by centrifugation and suspended in an aqueous buffer and fractured using a mechanical homogenization method by a French press, homogenizer or DYNO-MILL, freeze-thaw method, lysozyme addition method, ultrasonication method or surfactant addition method, either alone or in combination to give a cell-free extract, which subsequently is centrifuged to give a supernatant, from which the expression product may be purified using a ordinary method for isolating and purifying a protein, i.e., such as solvent extraction method, dialysis method, ultrafiltration method, gel filtration method, salt precipitation method with ammonium sulfate, etc., desalting method, precipitation method with an organic solvent, anion exchange chromatography method, cation exchange chromatography method, hydrophobic chromatography method, affinity chromatography method (e.g., metal chelate affinity chromatography method), chromatofocusing method, electrophoresis method (such as SDS-PAGE, agarose gel electrophoresis method, isoelectric point electrophoresis method), electroelution method, either alone or in combination.

In case when the expression product is accumulated within the cell as an insoluble form, the expression product may be purified as follows: the cells are collected, then fractured and centrifuged to collect the insoluble form of the polypeptide as a precipitated fraction, which is then solubilized with a protein denaturing agent, and the solution obtained is either diluted or dialyzed to bring the expression product back to its normal configuration before being subjected to isolation and purification methods as mentioned above.

In case when the expression product is secreted outside the cell, the expression product may be purified as follows: the culture is treated by centrifugation, etc. to give a soluble fraction, which is then subjected to isolation and purification methods as mentioned above.

In order to facilitate the collection of the expression product, a tag peptide having a binding ability to a particular substance may preliminarily be incorporated in the polypeptide to be expressed. Such tag peptide includes, such as, without limitation, His tag, GST tag, S tag and T7 tag. Thus, a subjected heterologous gene may comprise a nucleic acid sequence encoding for such a tag peptide.

The method of the present invention may be applied to various scale of production including from a small-scale production at a laboratory level to a large-scale production at a factory level. In each case, the aforementioned method of the present invention may appropriately be modified to be compatible to the scale. A modification compatible to each production scale is well known to those skilled in the art.

The present invention also relate to a kit for the production of a gene product, comprising a nucleic acid molecule, a nucleic acid construct, a vector and/or a host cell of the present invention.

The nucleic acid molecule, nucleic acid construct, vector and/or host cell of the present invention contained in the kit of the invention may be in any form as long as they exist in container(s) in a storable state, though, typically, they are stored in container(s) in form of a suspension in a storage solution or in a lyophilized form. When it is in a form of a suspension in a storage solution, a refrigerated or frozen storage may be necessary. The container preferably is sealed. Lyophilization may be carried out by any method known to those skilled in the art. Specifically, without limitation, for example, an object is suspended in a storage solution containing such as a dispersion medium, then the suspension is introduced into a sealable container (such as an ampule), which is then loaded into a lyophilizer for lyophilization. After finishing lyophilization, the container is sealed. Various operations such as production of the object, introduction into a container and lyophilization are carried out preferably under a sterile condition. Various storage solutions corresponding to the use and the object to be stored are known, and they may appropriately be used. Ingredients contained in a storage solution include, without limitation, e.g., propylene glycol, glycerol, polyethylene glycol, ethylene glycol, butanediol, formamide, propanediol, sorbitol, mannitol, DMSO, EDTA, Tris-HCl and TE (a mixture of Tris-HCl and EDTA).

A kit of the present invention may comprise one or more heterologous genes of interest as a nucleic acid construct operably linked to a nucleic acid molecule of the present invention or a vector comprising it, or alternatively may comprise a heterologous gene in another nucleic acid construct or vector other than the nucleic acid molecule of the present invention. Accordingly, the kit of the present invention may only comprise a nucleic acid construct or vector which comprises a nucleic acid molecule of the present invention but not a subject heterologous gene; or may only comprise a nucleic acid construct or vector which comprises a nucleic acid molecule of the present invention and a heterologous gene operably linked thereto; or may comprise a nucleic acid construct or vector which comprises a nucleic acid molecule of the present invention but not a subject heterologous gene and a nucleic acid construct or vector which comprises a subject heterologous gene but not a nucleic acid molecule of the present invention. A nucleic acid construct or vector which comprises a subject heterologous gene but not a nucleic acid molecule of the present invention may be provided separately as an optional item of the kit of the present invention. When a nucleic acid molecule of the present invention and a subject heterologous gene are comprised in separate nucleic acid constructs or vectors, each nucleic acid construct or vector may preferably be designed such that a nucleic acid construct or vector comprising a nucleic acid molecule of the present invention and a heterologous gene operably linked thereto is produced by treating these nucleic acid constructs or vectors with a restriction enzyme, etc.

A kit of the present invention may comprise elements useful for the production of a gene product other than those described above. Such elements include, without limitation, e.g., a reconstituent that reconstitutes the nucleic acid molecule, nucleic acid construct, vector and/or host cell of the present invention from the storage state, a restriction enzyme to be used for incorporation of the nucleic acid molecule, a culture medium for culturing the host cell, a selection medium for selecting the transformed host cell, a standard substance of the gene product of a known amount which will be a basis for assessing the expression level of the gene product, an instruction or an electric recording medium such as CDs and DVDs describing a reconstitution method for nucleic acid molecule, nucleic acid construct, vector and/or host cell of the present invention, and/or a process for the production of a gene product.

### EXAMPLES

Hereinbelow, the present invention is further described based on specific examples, though such specific examples are merely exemplifications of the present invention and do not limit the present invention.

### Example 1

### Cloning of C. perfringens ι-Toxin a Component (Ia) Gene

(1) Extraction of C. perfringens Plasmid DNA

Type E C. perfringens (NCIB10748) was cultured in 500 ml of the Brain Heart Infusion broth at 37° C. for 6 hours, centrifuged at 8,000 rpm to collect the cells. Pelleted cell bodies were suspended in TNE buffer (100 mM NaCl, 100 mM EDTA, 10 mM Tris-HCl (pH 8)), washed by centrifugation. 25% sucrose-containing TNE buffer and lysozyme (10 mg/ml) was added to the pelleted cell bodies, which was shaken at 37° C. for 15 minutes. Subsequently, EDTA was added to a final concentration of 100 mM, and the mixture was incubated at 37° C. for 20 minutes. To this, N-Sodium lauroyl sarcosinate (LSS) was added to a final concentration of 1% and gently stirred, and then the lysate was left overnight at 4° C. After centrifuging at 15,000 rpm for 20 minutes, the supernatant was collected, which was made to 8 ml with TNE buffer and 8 g of cesium chloride was dissolved therein, centrifuged using a vertical rotor at 4° C., 45,000 rpm for 12 hours, and a fraction containing plasmid DNAs was isolated while confirming with an ultraviolet lamp. Obtained fraction was dialyzed with 31 of TE buffer, then plasmid DNAs were purified by ethanol precipitation method, dissolved in TE buffer (1 mM EDTA, 10 mM Tris-HCl (pH 8.0)) to give a plasmid DNA as a starting material for cloning.

### (2) Cloning of Ia Gene

Using the DNA obtained as above as template, primers were generated referring to a known nucleotide sequence of Ia gene (Infect. Immun. 61, 5147-5156 (1993)), and a region comprising Ia structural gene and the upstream and downstream thereof was amplified by PCR. The primer sequences were as follows.

(SEQ ID NO: 25)
Forward primer: 5′-GAATTCAGAAAATACAATCT-3′
(SEQ ID NO: 26)
Reverse primer: 5′-TATGATAACGTTTGACTTAT-3′

Obtained PCR product of 1721 by (SEQ ID NO: 27) was inserted into pT7BlueT-Vector (Novagen) using Taq DNA polymerase to give a vector having Ia gene, i.e., pT-Ia.

### (3) Determination of the Nucleotide Sequence of Ia Gene

The obtained gene comprising the structural gene of Ia was about 1.3 kbp as confirmed by agarose gel electrophoresis. The whole sequence of pT-Ia was sequenced using Big dye reagent (Applied Biosystems) and the primers generated based on the Ia genetic sequence described in the reference above, and analyzed with ABI PRISM® 310 Genetic Analyzer (Applied Biosystems). The gene sequence of Ia was consistent with that described in the reference above.

### Example 2

### Construction of a Vector Comprising Ia Gene Promoter Region

From pT-Ia, a region containing the structural gene of Ia and the upstream and downstream thereof (SEQ ID NO: 27) was cut out with restriction enzymes EcoRI and XbaI, and inserted into a E. coli-B. subtilis shuttle vector pHY300PLK (TAKARA BIO INC.). The obtained vector pHY300PLK-CpIa, in which Ia gene has been inserted, contains a promoter region of Ia gene. In the downstream of this promoter region, i.e., between positions 1476 and 1477 of the complementary sequence of Ia gene sequence expressed by SEQ ID NO: 28, a multicloning sequence (SEQ ID NO: 29) was inserted. Specifically, an NdeI site (CATATG) was inserted into the above-mentioned site to generate a vector pHY300PLK-CpIa-NdeI, and then the above-mentioned multicloning site was cloned using Ligation kit (TAKARA BIO INC.). The obtained vector pHY300PLK-CpIa-MCS was subjected to the sequence analysis using ABI PRISM® 310 Genetic Analyzer (Applied Biosystems), confirming that a desired sequence (SEQ ID NO: 30) was obtained.

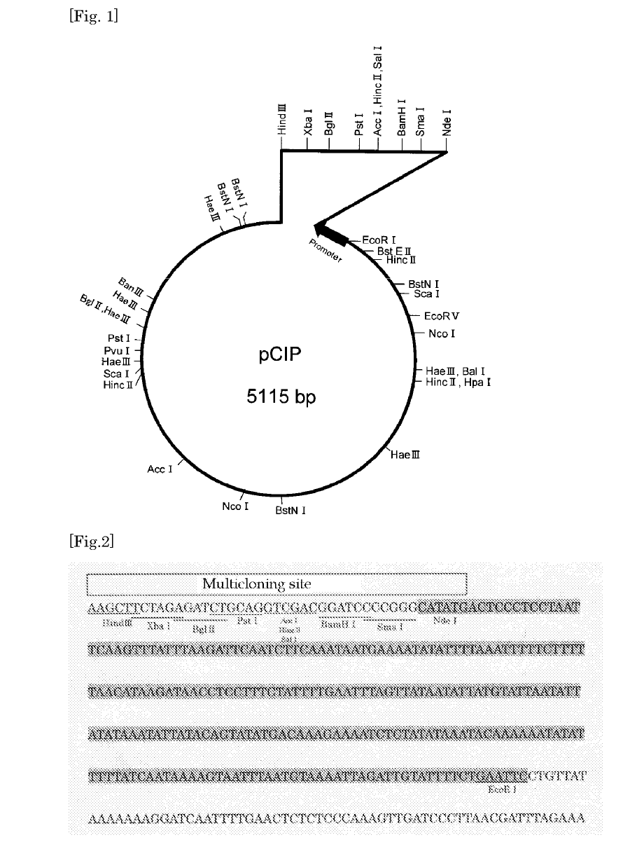

In another experiment, from pHY300PLK-CpIa-NdeI obtained as above, Ia gene promoter region (SEQ ID NO: 31) was cut out with NdeI and EcoRI, ligated into a modified pHY300PLK having a multicloning site with additional NdeI site on its 3′ terminal (SEQ ID NO: 32), constructing a vector pCIP containing Ia gene promoter region. The obtained pCIP was subjected to the sequence analysis using ABI PRISM® 310 Genetic Analyzer (Applied Biosystems), confirming that a desired sequence expressed by SEQ ID NO: 33 was obtained (see, FIGS. 1 and 2).

### Example 3

### Cloning of Heterologous Genes

(1) Cloning of C. perfringens α-Toxin Gene

1) Extraction of C. perfringens chromosomal DNA

Type A C. perfringens (NCTC8237) was cultured in 500 ml of the Brain Heart Infusion broth at 37° C. for 6 hours, centrifuged at 8,000 rpm to collect the cells. The cell bodies were suspended in TNE buffer, and washed by centrifugation. The cell bodies were suspended in TNE buffer, which was shaken at 37° C. for 60 minutes. To this, SDS was added to a final concentration of 1%, which was gently stirred, then an equal volume of saturated phenol-chloroform was added and the mixture was well mixed. After being centrifuged at 10,000 rpm for 10 minutes, the supernatant was collected. An equal volume of saturated chloroform-isoamyl alcohol was added to the supernatant and the mixture was well mixed, centrifuged at 10,000 rpm for 10 minutes, and the supernatant was collected and mixed with a double volume of 99% ethanol, and cooled at −30° C. for 2 hours to precipitate DNAs. Subsequently, after being centrifuged at 10,000 rpm for 10 minutes, the supernatant was discarded, and the precipitation was dried under reduced pressure for 20 minutes. This precipitation was dissolved in TE buffer, added thereto an RNase (Sigma) solution to be 125 mg/ml, and treated at 37° C. for 10 minutes. To this, NaCl was added to a final concentration of 0.1 M, and an equal volume of saturated chloroform-isoamyl alcohol was further added thereto, and the mixture was well stirred. After being centrifuged at 10,000 rpm for 10 minutes, 2.5 volumes of 99% ethanol was added to the supernatant, cooled at −80° C. for 30 minutes. The precipitated DNA was centrifuged at 10,000 rpm for 10 minutes, and then the precipitation was dried under reduced pressure, dissolved in TE buffer to give a chromosomal DNA as a starting material for cloning.

2) Cloning of C. perfringens α-Toxin Gene

The chromosomal DNA above was cleaved with HindIII, a DNA fragment of 3 to 4 kbp was collected and ligated with pUC19 vector which had been cleaved with HindIII and dephosphorylated. This ligation solution was used for transforming E. coli JM109, which was inoculated onto a yolk agar medium and cultured overnight. Colonies showing white turbidity in their periphery due to the expression of α-toxin gene were picked up, inoculated and cultured in an LB medium, then a plasmid contained α-toxin gene (pUA-3.1) was isolated.

3) Determination of the Nucleotide Sequence of C. perfringens α-Toxin Gene

The obtained gene containing the structural gene of α-toxin was about 3.1 kbp, as confirmed by agarose gel electrophoresis. The whole sequence of pUA-3.1 was sequenced using Big dye reagent (Applied Biosystems) and primers generated based on previously reported gene sequence of α-toxin (Infect. Immun. 57, 367-376 (1989)), analyzed using ABI PRISM® 310 Genetic Analyzer (Applied Biosystems). The obtained gene sequence of α-toxin was consistent with that described in the reference above.

4) Cloning of C. perfringens α-Toxin Gene into an Expression Vector

Using pUA-3.1 as template, a region of 1.3 kbp comprising the structural gene of α-toxin and the upstream and downstream thereof was amplified by PCR. The primer sequences were as follows.

(SEQ ID NO: 34)
Forward primer: 5′-TTTAAAAAATATTCAAAAAAT-3′
(SEQ ID NO: 35)
Reverse primer: 5′-GGAAGCTTTTATTTTGTAAATAC-3′

The obtained PCR product of 1.3 kbp (SEQ ID NO: 36) was made blunt-ended, inserted into pHY300PLK which had been cleaved with SmaI, to give C. perfringens α-toxin gene expression vector pHY300PLK-Cpα.

(2) Cloning of C. perfringens β2-Toxin Gene

1) Extraction of C. perfringens Plasmid DNA

Type A C. perfringens (Strain 13) was cultured in 500 ml of the Brain Heart Infusion broth at 37° C. for 6 hours, centrifuged at 8,000 rpm to collect the cells. Pelleted cell bodies were suspended in TNE buffer (100 mM NaCl, 100 mM EDTA, 10 mM Tris-HCl (pH 8)), and washed by centrifugation. 25% sucrose-containing TNE buffer and lysozyme (10 mg/ml) was added to the pelleted cell bodies, which was shaken at 37° C. for 15 minutes. Subsequently, EDTA was added to a final concentration of 100 mM, and the mixture was incubated at 37° C. for 20 minutes. To this, N-Sodium lauroyl sarcosinate (LSS) was added to a final concentration of 1% and gently stirred, and then the lysate was left overnight at 4° C. After centrifuging at 15,000 rpm for 20 minutes, the supernatant was collected, which was made to 8 ml with TNE buffer and 8 g of cesium chloride was dissolved therein, centrifuged using a vertical rotor at 4° C., 45,000 rpm for 12 hours, and a fraction containing plasmid DNAs was isolated while confirming with an ultraviolet lamp. Obtained fraction was dialyzed with 3 l of TE buffer, then plasmid DNAs were purified by ethanol precipitation method, dissolved in TE buffer (1 mM EDTA, 10 mM Tris-HCl (pH 8.0)) to give a plasmid DNA as a starting material for cloning.

2) Cloning of C. perfringens β2-Toxin Gene

Using the DNA obtained as above as template, primers were generated referring to a known nucleotide sequence of β2-toxin gene (Gene 203, 65-73 (1997)), (forward primer: 5′-TTAGATAAAAGTGTAAAAGA-3′ (SEQ ID NO: 37), reverse primer: 5′-TTAGGTTTTTATATAATAA-3′ (SEQ ID NO: 38)), and a region comprising the structural gene of β2-toxin and the upstream and downstream thereof was amplified by PCR. A PCR product of 960 bp (SEQ ID NO: 39) was inserted into pT7Blue vector to give a vector having β2-toxin gene, i.e., pT-132.

(5) Cloning of Bacillus cereus Sphingomyelinase (BcSMase) Gene

1) Extraction of B. cereus Plasmid DNA

B. cereus (IAM1029) was cultured in 500 ml of the Brain Heart Infusion broth at 37° C. for 6 hours, centrifuged at 8,000 rpm to collect the cells. Pelleted cell bodies were suspended in TNE buffer (100 mM NaCl, 100 mM EDTA, 10 mM Tris-HCl (pH 8)), and washed by centrifugation. 25% sucrose-containing TNE buffer and lysozyme (10 mg/ml) was added to the pelleted cell bodies, which was shaken at 37° C. for 15 minutes. Subsequently, EDTA was added to a final concentration of 100 mM, and the mixture was incubated at 37° C. for 20 minutes. To this, N-Sodium lauroyl sarcosinate (LSS) was added to a final concentration of 1% and gently stirred, then the lysate was left overnight at 4° C. After centrifuging at 15,000 rpm for 20 minutes, the supernatant was collected, which was made to 8 ml with TNE buffer and 8 g of cesium chloride was dissolved therein, centrifuged using a vertical rotor at 4° C., 45,000 rpm for 12 hours, a fraction containing plasmid DNAs was isolated while confirming with an ultraviolet lamp. Obtained fraction was dialyzed with 3 l of TE buffer, then plasmid DNAs were purified by ethanol precipitation method, dissolved in TE buffer (1 mM EDTA, 10 mM Tris-HCl (pH 8.0)) to give a plasmid DNA as a starting material for cloning.

2) Cloning of B. cereus Sphingomyelinase Gene

Using the DNA obtained as above as template, B. cereus sphingomyelinase gene was amplified by PCR using a forward primer: 5′-ATGGAGGTATGGAACGTG-3′ (SEQ ID NO: 40) and a reverse primer: 5′-CTACTTCATAGAAATAGT-3′ (SEQ ID NO: 41). A PCR product (SEQ ID NO: 42) was inserted into pT7Blue vector to give a vector having B. cereus sphingomyelinase gene, i.e., pT-BcSMase.

### Example 4

### Insertion of a Heterologous Gene into pCIP Vector

Into the multicloning site of pCIP generated in Example 2, a heterologous gene obtained in Example 3 (C. perfringens α-toxin gene, C. perfringens (32-toxin gene or B. cereus sphingomyelinase gene), C. botulinum α-toxin (phospholipase C (CbPLC)) gene or C. botulinum C3 enzyme gene was inserted.

(1) Insertion of C. perfringens α-Toxin Gene into pCIP

Using pUA-3.1 as template, a region of 1.3 kbp containing the structural gene of α-toxin and the upstream and downstream thereof was amplified by PCR. The primer sequences were as follows.

Forward primer:
(SEQ ID NO: 43)
5′-GGGCATATGATGAAAAGAAAGATTTGTAAG-3′
Reverse primer:
(SEQ ID NO: 44)
5′-CCCTCTAGATTATTTTATATTATAAGTTGA-3′

The obtained gene of about 1.2 kbp (SEQ ID NO: 45) was cleaved with NdeI and XbaI, ligated with pCIP that had been cleaved with the same enzymes, to give C. perfringens α-toxin gene expression vector pCIP-Cpα.

(2) Insertion of C. perfringens β2-Toxin Gene into pCIP

Using pT-β2 as template, a region of about 0.8 kbp containing the structural gene of β2-toxin and the upstream and downstream thereof was amplified by PCR. As primers, a forward primer having a leading NdeI restriction site and thereafter a sequence following the start codon: 5′-AGGCATATGAAAAAAATTATTTCAAAGTTT-3′ (SEQ ID NO: 46), and a reverse primer comprising the downstream 29 bps that terminates at a stop codon and having XbaI site at its end: 5′-CCTCTAGACTATGCACAATATCCTTC-3′ (SEQ ID NO: 47) were used. The obtained gene of about 0.8 kbp (SEQ ID NO: 48) was cleaved with NdeI and XbaI, ligated with pCIP that had been cleaved with the same enzymes, to give pCIP-Cpβ2.

(3) Insertion of B. cereus Sphingomyelinase Gene into pCIP

Using pT-BcSMase as template, a forward primer having a leading NdeI restriction site: 5′-CATATGATGGAGGTATGGAACGTG-3′ (SEQ ID NO: 49), and a reverse primer having an XbaI site at its end: 5′-TCTAGACTACTTCATAGAAATAGT-3′ (SEQ ID NO: 50) were used to amplify sphingomyelinase gene by PCR. The obtained gene of about 1.0 kb (SEQ ID NO: 51) was cleaved with NdeI and XbaI, ligated with pCIP that had been cleaved with the same enzyme to give pCIP-BcSMase.

(4) Insertion of C. botulinum Phospholipase C (CbPLC) Gene into pCIP

Using C. botulinum phospholipase C gene inserted into pUC18 vector (pUC18-HPLC, provided from Prof. Keiji Oguma of Okayama University, Graduate School) as template, a region of about 1.2 kbp containing the structural gene of CbPLC enzyme and the upstream and downstream thereof was amplified by PCR. As primers, a forward primer starting with a start codon and having a leading NdeI restriction site: 5′-CATATGATGAATAAGAAAAAAATATTAAAA-3′ (SEQ ID NO: 52), and a reverse primer having a stop codon and an XbaI site downstream thereto: 5′-TCTAGATTATTTATTATTTATATAGAATGT-3′ (SEQ ID NO: 53) were used. The obtained gene of about 1.2 kb (SEQ ID NO: 54) was cleaved with NdeI and XbaI, ligated with pCIP that had been cleaved with the same enzymes to give pCIP-CbPLC.

(5) Insertion of C. botulinum C3 Enzyme Gene into pCIP

Using C3 enzyme gene inserted into pUC18 vector (pUC18-C3, provided from Prof. Keiji Oguma of Okayama University, Graduate School) as template, a region of about 0.8 kbp containing the structural gene of C3 enzyme and the upstream and downstream thereof was amplified by PCR. As primers, a forward primer starting with a start codon and having a leading NdeI restriction site: 5′-GGCATATGAAAGGTTTAAGAAAATCA-3′ (SEQ ID NO: 55), and a reverse primer having a stop codon and an XbaI site downstream thereto: 5′-CCTCTAGATTATTTAGGATTGATAGCTGT-3′ (SEQ ID NO: 56) were used. The obtained gene of about 0.8 kb (SEQ ID NO: 57) was cleaved with NdeI and XbaI, ligated with pCIP that had been cleaved with the same enzymes to give pCIP-CbC3.

Each of the obtained vectors was sequenced with ABI PRISM® 310 Genetic Analyzer (Applied Biosystems), confirming that the desired sequences expressed by SEQ ID NOs: 58 to 62 were obtained.

### Example 5

### A Heterologous Gene Expression

(1) Culturing of the Host B. subtilis Cell

B. subtilis ISW1214 strain was pre-cultured overnight in 2 ml LB (Luria-Broth) medium at 37° C. 2 ml of the pre-culture was added to 32 ml of the main-culture (32 ml sterile LB medium supplemented with 2.9 g sorbitol and sterilized by an autoclave), and main culture was performed at 37° C. (about 3 hours). When the optical density (A620) reached from 0.85 to 0.95, the main culturing was ended, the culture was left in ice for 10 minutes and then centrifuged at 5,000×g (9500 rpm) for 5 minutes. Subsequently, the cell bodies were washed 4 times with an ice-cooled Solution A (0.5 M sorbitol+0.5 M mannitol+10% glycerol: 45.5 g sorbitol, 45.5 g mannitol and 50 ml glycerol were mixed, messed up with distilled water to 500 ml, then sterilized), then suspended in 0.8 ml (i.e., 1/40 volume of the culture solution) of Solution A. The obtained bacterial suspension was dispensed into sterile tubes (60 μA each) and stored at −80° C.

### (2) Introduction of a Vector

Each of the vectors generated in Examples 2 to 4 (pHY300PLK-CpIa, pHY300PLK-Cpa, pCIP-Cpα, pCIP-Cpβ2, pCIP-CbPLC, pCIP-CbC3 or pCIP-BcSMase) was introduced into a host B. subtilis cell by electroporation method. The electroporation was performed as follows. First, 60 μl of the bacterial suspension for electroporation prepared in (1) above was thawed in ice, and 1 μl vector solution (100 ng/μl) was added thereto. This mixture was transferred to an ice-cooled 0.1 cm cuvette and left for 1 to 1.5 minutes before pulsing (2 kV, 200Ω, 25 mF) by a gene-introducing device (BTX, ECM-630). Subsequently, this was transferred to a 15 ml centrifugation tube, 1 ml of Solution B (LB medium+0.5 M sorbitol+0.38 M mannitol) was added thereto, and cultured at 37° C. for 3 hours. The culture was centrifuged (3500 rpm, 5 minutes), 900 μl of the supernatant was removed, the pellet was suspended and plated onto a LB agar medium containing tetracycline (30 mg/ml), and cultured overnight at 37° C. Formed resistant colonies of the transformants were picked up, and subjected to the culturing as follows.

### (3) Culturing of the Transformant and Collection/Purification of the Gene Product

The transformant transfected with either of the vectors was cultured while stirring in 1l LB medium at 37° C. for 14 hours, then centrifuged at 4° C., 8,000 rpm for 20 minutes. To the culture supernatant, ammonium sulfate (Nacalai Tesque, 02619-86) was added at regular intervals in small amount while ice cooling and stirring to make a saturated ammonium sulfate (472 g/l) of a final concentration of 70%, which was left overnight. It was then centrifuged at 4° C., 9,500 rpm for 30 minutes, the resulting pellet was dissolved in 0.02 M TB buffer (0.02 M Tris/HCl, pH 7.5), dialyzed overnight with the same buffer at 4° C. After dialysis, it was centrifuged at 4° C., 15,000 rpm for 30 minutes to give a supernatant as a crude product (ammonium sulfate product) sample. This crude product sample was diluted with 1 M NaCl-TB (pH 7.5) to make the final concentration of NaCl to be 0.5M, then purified using copper-chelating affinity column (Chelating Sepharose Fast Flow, GE healthcare), anion exchange column (UNO™ Q-1 R Column, BIO-RAD, 720-0011) and/or cation exchange column (SP-TOYOPEARL 650 M, Tosoh Corporation). Each of the obtained products was measured for the protein content by BCA method and confirmed for the absence of impurity by SDS-PAGE. A comparison of the final amount purified from 1 l of culture and the maximum final amount purified from 1 l of culture using similar procedures using conventional pHY300PLK is shown below.

TABLE 1

| | Purification | | Conventional |
|---|---|---|---|
| Vector name | column | Yield (mg) | yield (mg) |
| pHY300PLK-CpIa | P | 42.8 | — |
| pCIP-Cpα | Cu→N | 48.5 | 12 |
| pCIP-Cpβ2 | Cu→N | 25.3 | 8 |
| pCIP-CbPLC | Cu→N | 23.7 | 3 |
| pCIP-CbC3 | P | 45.6 | 5 |
| pCIP-BcSMase | Cu→N | 58.6 | 5 |

Purification column: P (cation exchange column), N (anion exchange column), Cu (copper-chelating affinity column)

The results described above indicate that a vector of the present invention comprising a promoter region of C. perfringens ι-toxin gene significantly increases the heterologous gene expression. Also, since C. perfringens a and 132-toxin gene were highly expressed in B. subtilis cell using a conventional vector, it was suggested that the promoter of said gene may increase the heterologous gene expression.

1.-2. (canceled)

3. A nucleic acid construct that expresses a heterologous gene in a Bacillus bacterium cell, comprising a nucleic acid molecule comprising a promoter region derived from a Clostridium toxin gene and the heterologous gene operably linked thereto.

4.-8. (canceled)

9. The nucleic acid construct according to claim 3, wherein the toxin is selected from the group consisting of C. perfringens α-toxin, ε-toxin and ι-toxin, botulinum toxin, and tetanus toxin.

10. A vector that expresses a heterologous gene in a Bacillus bacterium cell, comprising the nucleic acid construct according to claim 3.

11.-12. (canceled)

13. A Bacillus bacterium host cell transformed with the vector according to claim 10.

14. The host cell according to claim 13, wherein the Bacillus bacterium is Bacillus subtilis.

15. A process for the production of a gene product, comprising a step of culturing the host cell according to claim 13.

16. A kit for the production of a gene product in a Bacillus bacterium cell, comprising the nucleic acid construct according to claim 3.

17.-18. (canceled)

19. The nucleic acid construct according to claim 3, wherein the toxin is C. perfringens ι-toxin.

20. A kit for the production of a gene product in a Bacillus bacterium cell, comprising the vector according to claim 10.

21. A kit for the production of a gene product in a Bacillus bacterium cell, comprising the host cell according to claim 13.

22. A kit comprising:
(i) a vector comprising a nucleic acid molecule comprising a promoter region derived from a Clostridium toxin gene and a heterologous gene operably linked thereto, or a combination of a vector comprising a nucleic acid molecule comprising a promoter region derived from a Clostridium toxin gene and a vector comprising a heterologous gene, and
(ii) a Bacillus bacterium cell.

23. The kit according to claim 22, wherein the Bacillus bacterium is Bacillus subtilis.

24. The kit according to claim 22, wherein the toxin is C. perfringens ι-toxin.

25. A method of producing a gene product in a Bacillus bacterium cell, comprising a step of introducing to said cell the nucleic acid construct according to claim 3.

26. A method of producing a gene product in a Bacillus bacterium cell, comprising a step of introducing to said cell the vector according to claim 10.

27. A method of producing a gene product in a Bacillus bacterium cell, comprising a step of:
(i) preparing a nucleic acid construct or a vector comprising a nucleic acid molecule comprising a promoter region derived from a Clostridium toxin gene and a heterologous gene operably linked thereto, and
(ii) introducing said nucleic acid construct or a vector to the Bacillus bacterium cell.

28. The method according to claim 27, wherein the production of the gene product has been enhanced compared to that of the case when pHY300PLK vector was used.

29. The method according to claim 27, wherein the Bacillus bacterium is Bacillus subtilis.


SEQUENCE LISTING (62 sequences)

SEQ ID NO: 1 - DNA, 784 bp, Clostridium perfringens
gaattccaagaccatcgataccttcacttattatatattctctaacgataggtgcaagggcagcctttccatctccacctcttgaatagattgttctaccagacccttttaattaacatcatatcttttactatctttagttacatgttctcctaataagagtgctctaccatctcctagcattgtaaaatgaccaaattggtcgcccagcataagcctgtgcaattggtacaatcctggaagttcattcctgcaaatatattaagtccaaaatcgctgtttaaaacttcttcattaagcccaagttcttcagcaagagaagtattaaacttaataagtttaggattttttgaaccctttggattttgttcactaaagaatatatttggaagagttaaataagtgttttctaagttaaaacctgtttttgattgaaaatttttattatccatattaaaatcctttgccttataatttatttcaaattttatccatcccttatattatgtgtaaaaattcttattaaattaaaaaacaagatttaacttattatagcactaataattgtaaattttcatattaaaaataagtttaacaatttagagtgggtaaggttagatatgtttaattgaaatttgaattgtattcaaaaatattttaaaaaatattcaaaaatttagtgagcttatggtaattatatggtataatttcagtggcaagtgttaatcggttatcaaaaaaggggagattaatacttgaaaaaaattaacgggggatataaaaa

SEQ ID NO: 2 - DNA, 187 bp, Clostridium perfringens
gatcgtttttagttctatttaaataaacgatttaataataaaaatttttaacttgggttttgtcgtaaatgttggagctaccccaatataataaaatttgtatattaaataattttatttatattatttactttttttaaaaaatatagaaaaatatagaaaaatatattaatgaaagggtggtttt

SEQ ID NO: 3 - DNA, 245 bp, Clostridium perfringens
gaattcagaaaatacaatctaattttacattaaattacttttattgataaaaatatattttttgtatttatatagagattttctttgtcatatactgtataatatttatataatattaatacataatattataactaaattcaaaatagaaaggaggttatcttatgttaaaaagaaaaatttaaaatatattttcattatttgaagattgaatcttaaataaacttgaattaggagggagtatt

SEQ ID NO: 4 - DNA, 76 bp, Clostridium botulinum
tcaaagtatttgtatttatggtcatttaaataattaataatttaattaattttaaatattataagaggtgttaaat

SEQ ID NO: 5 - DNA, 227 bp, Clostridium botulinum
gaattcaactagtagataacaaaaataatgcacagatttttattattaataatgatatatttatctctaactgtttaactttaacttataacaatgtaaatgtatatttgtctataaaaaatcaagattacaattgggttatatgtgatcttaatcatgatataccaaaaaagtcatatctatggatattaaaaaatatataaatttaaaattaggagatgctgtat

SEQ ID NO: 6 - DNA, 280 bp, Clostridium tetani
tagcattaaaaaaattagaacctatagtaaataaattaattaatatatagtttttataatttaattatgaataatattcttaagataaaaagtaaatttttaaaaatttaaattttcagtttacaaaaaataacctgattatgttatatgtaattgtaaaaaacatataaaaaatcagaaaaatttaggaggtatattattaatggattaaataataattttttaatttacttttgattaataaatattaaatgtttattttaattaggagatgatacgt

SEQ ID NO: 7 - DNA, 29 bp, Clostridium perfringens
tgagcttatggtaattatatggtataatt

SEQ ID NO: 8 - DNA, 29 bp, Clostridium perfringens
tttgtatattaaataattttatttatatt

SEQ ID NO: 9 - DNA, 30 bp, Clostridium perfringens
ttgtcatatactgtataatatttatataat

SEQ ID NO: 10 - DNA, 28 bp, Clostridium botulinum
tggtcatttaaataattaataatttaat

SEQ ID NO: 11 - DNA, 6 bp, Clostridium botulinum
tatatt

SEQ ID NO: 12 - DNA, 8 bp, Clostridium tetani
tatattat

SEQ ID NO: 13 - DNA, 104 bp, Clostridium perfringens
tgagcttatggtaattatatggtataatttcagtggcaagtgttaatcggttatcaaaaaaggggagattaatacttgaaaaaaattaacgggggatataaaaa

SEQ ID NO: 14 - DNA, 91 bp, Clostridium perfringens
tttgtatattaaataattttatttatattatttactttttttaaaaaatatagaaaaatatagaaaaatatattaatgaaagggtggtttt

SEQ ID NO: 15 - DNA, 161 bp, Clostridium perfringens
ttgtcatatactgtataatatttatataatattaatacataatattataactaaattcaaaatagaaaggaggttatcttatgttaaaaagaaaaatttaaaatatattttcattatttgaagattgaatcttaaataaacttgaattaggagggagtatt

SEQ ID NO: 16 - DNA, 58 bp, Clostridium botulinum
tggtcatttaaataattaataatttaattaattttaaatattataagaggtgttaaat

SEQ ID NO: 17 - DNA, 125 bp, Clostridium botulinum
tatatttgtctataaaaaatcaagattacaattgggttatatgtgatcttaatcatgatataccaaaaaagtcatatctatggatattaaaaaatatataaatttaaaattaggagatgctgtat

SEQ ID NO: 18 - DNA, 88 bp, Clostridium tetani
tatattattaatggattaaataataattttttaatttacttttgattaataaatattaaatgtttattttaattaggagatgatacgt

SEQ ID NO: 19 - DNA, 95 bp, Clostridium perfringens
tgagcttatggtaattatatggtataatttcagtggcaagtgttaatcggttatcaaaaaaggggagattaatacttgaaaaaaattaacggggg

SEQ ID NO: 20 - DNA, 87 bp, Clostridium perfringens
tttgtatattaaataattttatttatattatttactttttttaaaaaatatagaaaaatatagaaaaatatattaatgaaagggtgg

SEQ ID NO: 21 - DNA, 154 bp, Clostridium perfringens
ttgtcatatactgtataatatttatataatattaatacataatattataactaaattcaaaatagaaaggaggttatcttatgttaaaaagaaaaatttaaaatatattttcattatttgaagattgaatcttaaataaacttgaattaggagg

SEQ ID NO: 22 - DNA, 50 bp, Clostridium botulinum
tggtcatttaaataattaataatttaattaattttaaatattataagagg

SEQ ID NO: 23 - DNA, 117 bp, Clostridium botulinum
tatatttgtctataaaaaatcaagattacaattgggttatatgtgatcttaatcatgatataccaaaaaagtcatatctatggatattaaaaaatatataaatttaaaattaggaga

SEQ ID NO: 24 - DNA, 80 bp, Clostridium tetani
tatattattaatggattaaataataattttttaatttacttttgattaataaatattaaatgtttattttaattaggaga

SEQ ID NO: 25 - DNA, 20 bp, Artificial sequence
  CpIa forward primer
gaattcagaaaatacaatct

SEQ ID NO: 26 - DNA, 20 bp, Artificial sequence
  CpIa reverse primer
tatgataacgtttgacttat

SEQ ID NO: 27 - DNA, 1721 bp, Clostridium perfringens
gaattcagaaaatacaatctaattttacattaaattacttttattgataaaaatatattttttgtatttatatagagattttctttgtcatatactgtataatatttatataatattaatacataatattataactaaattcaaaatagaaaggaggttatcttatgttaaaaagaaaaatttaaaatatattttcattatttgaagattgaatcttaaataaacttgaattaggagggagtattatgaaaaaagttaataaatctatatctgtatttctaatattatatttaattttaactagttcatttcctagttatacttatgcacaagatttacaaatagcaagcaattatattacagatagagcttttattgaaagaccagaagattttcttaaagataaagaaaatgctattcaatgggaaaaaaaggaggctgaaagagtagaaaaaaaccttgatacacttgaaaaagaagcattagaattatataaaaaagattctgaacaaataagtaactactctcagacaagacagtatttttacgactatcaaatagaatcaaatcctagagaaaaagaatacaaaaatcttagaaatgccatatcaaaaaataagatagataaacctataaatgtttattattttgagtctccagagaaatttgcgtttaataaagaaataagaacagaaaatcaaaatgaaatttctttagagaaatttaatgagttgaaagaaactattcaagataaattgtttaaacaagatggatttaaggatgtttctttatatgaaccaggtaatggcgatgaaaagcctacaccactacttatacatttgaaattaccaaaaaatactggtatgttaccatatataaattctaatgatgtaaaaacattaatagaacaagactatagcataaagatagacaaaattgttcgtatagtaatagaaggaaagcaatatataaaagctgaagcttctattgtaaacagtcttgattttaaagatgatgtaagtaaaggtgatttatggggaaaagaaaattatagtgattggagtaataaattaactcctaatgaacttgctgatgtaaatgactatatgcgtggaggatataccgcaattaataactatttaatatcaaatggtcctttaaataatcctaatccagaactagactctaaagtaaataacattgaaaacgcattaaagctcacacctattccatctaacttaattgtatatagaaggtctggtccacaagaatttggattaactctcacatctcctgaatatgattttaataaaatagaaaatatagatgcttttaaagaaaaatgggaaggaaaagtaataacatacccaaactttattagtactagtattggaagtgtaaatatgagtgcatttgctaaaagaaaaataatactacgtataaacataccaaaagattctccaggagcttatttatcagccattccaggttatgcaggagaatatgaagtacttttaaatcatggaagtaaatttaaaatcaataaagttgattcttataaagatggaactgtaacaaaactaattttggatgcaacattgataaattaatacttttaatataaataatcaatttaaaggaggaaaaataatgaatatacaaattaaaaatgtatttagttttttaacacttacagctatgataagtcaaacgttatcata

SEQ ID NO: 28 - DNA, 1721 bp, Clostridium perfringens
tatgataacgtttgacttatcatagctgtaagtgttaaaaaactaaatacatttttaatttgtatattcattatttttcctcctttaaattgattatttatattaaaagtattaatttatcaatgttgcatccaaaattagttttgttacagttccatctttataagaatcaactttattgattttaaatttacttccatgatttaaaagtacttcatattctcctgcataacctggaatggctgataaataagctcctggagaatcttttggtatgtttatacgtagtattatttttcttttagcaaatgcactcatatttacacttccaatactagtactaataaagtttgggtatgttattacttttccttcccatttttctttaaaagcatctatattttctattttattaaaatcatattcaggagatgtgagagttaatccaaattcttgtggaccagaccttctatatacaattaagttagatggaataggtgtgagctttaatgcgttttcaatgttatttactttagagtctagttctggattaggattatttaaaggaccatttgatattaaatagttattaattgcggtatatcctccacgcatatagtcatttacatcagcaagttcattaggagttaatttattactccaatcactataattttcttttccccataaatcacctttacttacatcatctttaaaatcaagactgtttacaatagaagcttcagcttttatatattgctttccttctattactatacgaacaattttgtctatctttatgctatagtcttgttctattaatgtttttacatcattagaatttatatatggtaacataccagtattttttggtaatttcaaatgtataagtagtggtgtaggcttttcatcgccattacctggttcatataaagaaacatccttaaatccatcttgtttaaacaatttatcttgaatagtttctttcaactcattaaatttctctaaagaaatttcattttgattttctgttcttatttctttattaaacgcaaatttctctggagactcaaaataataaacatttataggtttatctatcttattttttgatatggcatttctaagatttttgtattctttttctctaggatttgattctatttgatagtcgtaaaaatactgtcttgtctgagagtagttacttatttgttcagaatcttttttatataattctaatgcttctttttcaagtgtatcaaggtttttttctactctttcagcctcctttttttcccattgaatagcattttctttatctttaagaaaatcttctggtctttcaataaaagctctatctgtaatataattgcttgctatttgtaaatcttgtgcataagtataactaggaaatgaactagttaaaattaaatataatattagaaatacagatatagatttattaacttttttcataatactccctcctaattcaagtttatttaagattcaatcttcaaataatgaaaatatattttaaatttttctttttaacataagataacctcctttctattttgaatttagttataatattatgtattaatattatataaatattatacagtatatgacaaagaaaatctctatataaatacaaaaaatatatttttatcaataaaagtaatttaatgtaaaattagattgtattttctgaattc

SEQ ID NO: 29 - DNA, 42 bp, Artificial sequence
  MCS for pHY300PLK-Ia-MCS
catatgcccgggggatccgtcgacctgcagagatcttctaga

SEQ ID NO: 30 - DNA, 6626 bp, Artificial sequence
  pHY300PLK-Ia-MCS
aagcttctagaggatctactagtcatatggatttatgataacgtttgacttatcatagctgtaagtgttaaaaaactaaatacatttttaatttgtatattcattatttttcctcctttaaattgattatttatattaaaagtattaatttatcaatgttgcatccaaaattagttttgttacagttccatctttataagaatcaactttattgattttaaatttacttccatgatttaaaagtacttcatattctcctgcataacctggaatggctgataaataagctcctggagaatcttttggtatgtttatacgtagtattatttttcttttagcaaatgcactcatatttacacttccaatactagtactaataaagtttgggtatgttattacttttccttcccatttttctttaaaagcatctatattttctattttattaaaatcatattcaggagatgtgagagttaatccaaattcttgtggaccagaccttctatatacaattaagttagatggaataggtgtgagctttaatgcgttttcaatgttatttactttagagtctagttctggattaggattatttaaaggaccatttgatattaaatagttattaattgcggtatatcctccacgcatatagtcatttacatcagcaagttcattaggagttaatttattactccaatcactataattttcttttccccataaatcacctttacttacatcatctttaaaatcaagactgtttacaatagaagcttcagcttttatatattgctttccttctattactatacgaacaattttgtctatctttatgctatagtcttgttctattaatgtttttacatcattagaatttatatatggtaacataccagtattttttggtaatttcaaatgtataagtagtggtgtaggcttttcatcgccattacctggttcatataaagaaacatccttaaatccatcttgtttaaacaatttatcttgaatagtttctttcaactcattaaatttctctaaagaaatttcattttgattttctgttcttatttctttattaaacgcaaatttctctggagactcaaaataataaacatttataggtttatctatcttattttttgatatggcatttctaagatttttgtattctttttctctaggatttgattctatttgatagtcgtaaaaatactgtcttgtctgagagtagttacttatttgttcagaatcttttttatataattctaatgcttctttttcaagtgtatcaaggtttttttctactctttcagcctcctttttttcccattgaatagcattttctttatctttaagaaaatcttctggtctttcaataaaagctctatctgtaatataattgcttgctatttgtaaatcttgtgcataagtataactaggaaatgaactagttaaaattaaatataatattagaaatacagatatagatttattaacttttttcatcatatgcccgggggatccgtcgacctgcagagatcttctagaaatactccctcctaattcaagtttatttaagattcaatcttcaaataatgaaaatatattttaaatttttctttttaacataagataacctcctttctattttgaatttagttataatattatgtattaatattatataaatattatacagtatatgacaaagaaaatctctatataaatacaaaaaatatatttttatcaataaaagtaatttaatgtaaaattagattgtattttctgaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccc

SEQ ID NO: 31 - DNA, 248 bp, Artificial sequence
  CpIa promoter insert
catatgactccctcctaattcaagtttatttaagattcaatcttcaaataatgaaaatatattttaaatttttctttttaacataagataacctcctttctattttgaatttagttataatattatgtattaatattatataaatattatacagtatatgacaaagaaaatctctatataaatacaaaaaatatatttttatcaataaaagtaatttaatgtaaaattagattgtattttctgaattc

SEQ ID NO: 32 - DNA, 37 bp, Artificial sequence
  MCS for pCIP
aagcttctagagatctgcaggtcgacggatccccggg

SEQ ID NO: 33 - DNA, 5115 bp, Artificial sequence
  pCIP
aagcttctagagatctgcaggtcgacggatccccgggcatatgactccctcctaattcaagtttatttaagattcaatcttcaaataatgaaaatatattttaaatttttctttttaacataagataacctcctttctattttgaatttagttataatattatgtattaatattatataaatattatacagtatatgacaaagaaaatctctatataaatacaaaaaatatatttttatcaataaaagtaatttaatgtaaaattagattgtattttctgaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccc

SEQ ID NO: 34 - DNA, 21 bp, Artificial sequence
  Forward primer for pHY300PLK-CpA
tttaaaaaatattcaaaaaat

SEQ ID NO: 35 - DNA, 23 bp, Artificial sequence
  Reverse primer for pHY300PLK-CpA
ggaagcttttattttgtaaatac

SEQ ID NO: 36 - DNA, 1385 bp, Clostridium perfringens
aaaaatattttaaaaaatattcaaaaatttagtgaggttatggtaattatatggtataatttcagtgcaagtgttaatcgttatcaaaaaaggggagattaatacttgaaaaaaaataacgggggatataaaaatgaaaagaaagatttgtaaggcgcttatttgtgctacgctagcaactagcctatgggctggggcatcaactaaagtctacgcttgggatggaaagattgatggaacaggaactcatgctatgattgtaactcaaggggtttcaatcttagaaaatgatctgtccaaaaatgaaccagaaagtgtaagaaaaaacttagagattttaaaagagaacatgcatgagcttcaattaggttctacttatccagattatgataagaatgcatatgatctatatcaagatcatttctgggatcctgatacagataataatttctcaaaggataatagttggtatttagcttattctatacctgacacaggggaatcacaaataagaaaattttcagcattagctagatatgaatggcaaagaggaaactataaacaagctacattctatcttggagaggctatgcactattttggagatatagatactccatatcatcctgctaatgttactgccgttgatagcgcaggacatgttaagtttgagacttttgcagaggaaagaaaagaacagtataaaataaacacagcaggttgcaaaactaatgaggatttttatgctgatatcttaaaaaacaaagattttaatgcatggtcaaaagaatatgcaagaggttttgctaaaacaggaaaatcaatatactatagtcatgctagcatgagtcatagttgggatgattgggattatgcagcaaaggtaactttagctaactctcaaaaaggaacagcaggatatatttatagattcttacacgatgtatcagagggtaatgatccatcagttggaaagaatgtaaaagaactagtagcttacatatcaactagtggtgagaaagatgctggaacagatgactacatgtattttggaatcaaaacaaaggatggaaaaactcaagaatgggaaatggacaacccaggaaatgattttatgactggaagtaaagacacttatactttcaaattaaaagatgaaaatctaaaaattgatgatatacaaaatatgtggattagaaaaagaaaatatacagcattcccagatgcttataagccagaaaacataaagataatagcaaatggaaaagttgtagtagacaaagatataaacgagtggatttcaggaaattcaacttataatataaaataataaaagtaaaaaaataattattggttttggtggtatttacaaaataaaagcttcc

SEQ ID NO: 37 - DNA, 20 bp, Artificial sequence
  Forward primer for CpB2
ttagataaaagtgtaaaaga

SEQ ID NO: 38 - DNA, 19 bp, Artificial sequence
  Reverse primer for CpB2
ttaggtttttatataataa

SEQ ID NO: 39 - DNA, 960 bp, Clostridium perfringens
ttagataaaagtgtaaaagaattatttttattttaaatttgttaaaattttgatataattgaattgtaaaaaaaatttcaggggggaatataaatgaaaaaaattatttcaaagtttactgtaatttttatgttttcatattttcttattgttggagcaataagtccaatgaaagcaagtgcaaaggaaatcgacgcttatagaaaggtaatggagaattatcttaatgcttttaaaaactacgatattaatacgattgtaaacgtatcagaagatgaaagagtgaatagtgatgaaaagtataaagagatgttagaagagtttaaatatgatcctaaccaacaactaaaatcttttgaaatacttaattcacaaaagattgataataaagaaatatttaatgtaaaaactgaatttatgaatggtgcaatttatgatatgaaatttactgtatcatctaaagatggggaattaatagtatctgacatggaaagaacaaaaattgagaatgagggaaaatatattttaacaccatcatttagaactcaagtttgtacatgggatgatgaattatcacaatcaattgggggagttgatccaaaaacatattctactagatttacatattatgcagacaatatattattaaactttagacaatatgcaacttcaggttcaagagatttaaaagtagaatatagtgttgtagatcattggttatggggagatgatgttaaagcttctcaaatggtgtatggtcaaaaccctgattctgctagacaaataagattatatatagaaaaaggacaatctttctataaatatagaataagaatacaaaactttacacctgcatcaattagagtatttggtgaaggatattgtgcatagaaaaaaatatgaagtgactaagtcacttcatattttttttactattaattttattatataaaaacctaa

SEQ ID NO: 40 - DNA, 18 bp, Artificial sequence
  Forward primer for BcSMase
atggaggtatggaacgtg

SEQ ID NO: 41 - DNA, 18 bp, Artificial sequence
  Reverse primer for BcSMase
ctacttcatagaaatagt

SEQ ID NO: 42 - DNA, 1017 bp, Bacillus cereus
atggaggtatggaacgtgaaatgtaaattgctaaaaggtgtacttagcttaggtgttggtttaggagctttatatagcggaacgtcagctcaagcagaagtgtctacaaatcaaaatgatacattaaaagtgatgacgcataatgtatatatgctatcaacaaacttatatccgaactggggacaaactgagcgtgctgatttaatcggggcggcagattatataaagaatcaagatgtagttatattaaatgaagtgtttgataatagcgcttcagatcgtttattagggaatttgaagaaagaatatccaaatcaaacagcagtattaggtcgtagtagtggaagtgaatgggataaaacgttaggaaactattcatcttcaactcctgaagatggtggcgttgcgattgtgagcaaatggccaatcgctgaaaagattcaatatgtatttgcaaaaggatgcgggccagataatttatcgaataaaggatttgtatacacgaaaattaagaaaaatgatcgcttcgttcacgtgattggtacacatttgcaagcggaagatagtatgtgcggaaaaacttcacctgcatctgtacgtacagaccaattaaaagaaattcaagactttattaaaaataaaaatataccaaataatgagtatgtgttaattggcggtgatatgaacgtaaataaaattaatgcagagaacaagaatgattcagagtatgcatctatgtttaaaacattgaacgcttctgtaccatcttatactggacatacagcgacttgggacgcaacgacaaacagtattgcaaaatataatttccctgatagtcctgctgagtatttagattatattattgcaagtaaagaccatgcgaacccatcatatatagagaataaggtgttacagccgaaatctccacaatggactgttacatcgtggttccaaaaatatacgtataatgattactctgatcattatccagtagaggcgactatttctatgaagtag

SEQ ID NO: 43 - DNA, 30 bp, Artificial sequence
  Forward primer for pCIP-CpA
gggcatatgatgaaaagaaagatttgtaag

SEQ ID NO: 44 - DNA, 30 bp, Artificial sequence
  Reverse primer for pCIP-CpA
ccctctagattattttatattataagttga

SEQ ID NO: 45 - DNA, 1212 bp, Artificial sequence
  Insert for pCIP-CpA
gggcatatgaaaagaaagatttgtaaggcgcttatttgtgccgcgctagcaactagcctatgggctggggcatcaactaaagtctacgcttgggatggaaagattgatggaacaggaactcatgctatgattgtaactcaaggggtttcaatcttagaaaatgatctgtccaaaaatgaaccagaaagtgtaagaaaaaacttagagattttaaaagagaacatgcatgagcttcaattaggttctacttatccagattatgataagaatgcatatgatctatatcaagatcatttctgggatcctgatacagataataatttctcaaaggataatagttggtatttagcttattctatacctgacacaggggaatcacaaataagaaaattttcagcattagctagatatgaatggcaaagaggaaactataaacaagctacattctatcttggagaggctatgcactattttggagatatagatactccatatcatcctgctaatgttactgccgttgatagcgcaggacatgttaagtttgagacttttgcagaggaaagaaaagaacagtataaaataaacacagcaggttgcaaaactaatgaggctttttatactgatatcttaaaaaacaaagattttaatgcatggtcaaaagaatatgcaagaggttttgctaaaacaggaaaatcaatatactatagtcatgctagcatgagtcatagttgggatgattgggattatgcagcaaaggtaactttagctaactctcaaaaaggaacagcgggatatatttatagattcttacacgatgtatcagagggtaatgatccatcagttggaaagaatgtaaaagaactagtagcttacatatcaactagtggtgagaaagatgctggaacagatgactacatgtattttggaatcaaaacaaaggatggaaaaactcaagaatgggaaatggacaacccaggaaatgattttatgactggaagtaaagacacttatactttcaaattaaaagatgaaaatctaaaaattgatgatatacaaaatatgtggattagaaaaagaaaatatacagcattctcagatgcttataagccagaaaacataaagataatagcaaatggaaaagttgtagtggacaaagatataaacgagtggatttcaggaaattcaacttataatataaaataatctagaggg

SEQ ID NO: 46 - DNA, 30 bp, Artificial sequence
  Forward primer for pCIP-CpB2
aggcatatgaaaaaaattatttcaaagttt

SEQ ID NO: 47 - DNA, 26 bp, Artificial sequence
  Reverse primer for pCIP-CpB2
cctctagactatgcacaatatccttc

SEQ ID NO: 48 - DNA, 812 bp, Artificial sequence
  Insert for pCIP-CpB2
aggcatatgaaaaaaattatttcaaagtttactgtaatttttatgttttcatattttcttattgttggagcaataagtccaatgaaagcaagtgcaaaggaaatcgacgcttatagaaaggtaatggagaattatcttaatgcttttaaaaactacgatattaatacgattgtaaacgtatcagaagatgaaagagtgaatagtgatgaaaagtataaagagatgttagaagagtttaaatatgatcctaaccaacaactaaaatcttttgaaatacttaattcacaaaagattgataataaagaaatatttaatgtaaaaactgaatttatgaatggtgcaatttatgatatgaaatttactgtatcatctaaagatggggaattaatagtatctgacatggaaagaacaaaaattgagaatgagggaaaatatattttaacaccatcatttagaactcaagtttgtacatgggatgatgaattatcacaatcaattgggggagttgatccaaaaacatattctactagatttacatattatgcagacaatatattattaaactttagacaatatgcaacttcaggttcaagagatttaaaagtagaatatagtgttgtagatcattggttatggggagatgatgttaaagcttctcaaatggtgtatggtcaaaaccctgattctgctagacaaataagattatatatagaaaaaggacaatctttctataaatatagaataagaatacaaaactttacacctgcatcaattagagtatttggtgaaggatattgtgcatagtctagagg

SEQ ID NO: 49 - DNA, 24 bp, Artificial sequence
  Forward primer for pCIP-BcSMase
catatgatggaggtatggaacgtg

SEQ ID NO: 50 - DNA, 24 bp, Artificial sequence
  Reverse primer for pCIP-BcSMase
tctagactacttcatagaaatagt

SEQ ID NO: 51 - DNA, 1029 bp, Artificial sequence
  Insert for pCIP-BcSMase
catatgatggaggtatggaacgtgaaatgtaaattgctaaaaggtgtacttagcttaggtgttggtttaggagctttatatagcggaacgtcagctcaagcagaagtgtctacaaatcaaaatgatacattaaaagtgatgacgcataatgtatatatgctatcaacaaacttatatccgaactggggacaaactgagcgtgctgatttaatcggggcggcagattatataaagaatcaagatgtagttatattaaatgaagtgtttgataatagcgcttcagatcgtttattagggaatttgaagaaagaatatccaaatcaaacagcagtattaggtcgtagtagtggaagtgaatgggataaaacgttaggaaactattcatcttcaactcctgaagatggtggcgttgcgattgtgagcaaatggccaatcgctgaaaagattcaatatgtatttgcaaaaggatgcgggccagataatttatcgaataaaggatttgtatacacgaaaattaagaaaaatgatcgcttcgttcacgtgattggtacacatttgcaagcggaagatagtatgtgcggaaaaacttcacctgcatctgtacgtacagaccaattaaaagaaattcaagactttattaaaaataaaaatataccaaataatgagtatgtgttaattggcggtgatatgaacgtaaataaaattaatgcagagaacaagaatgattcagagtatgcatctatgtttaaaacattgaacgcttctgtaccatcttatactggacatacagcgacttgggacgcaacgacaaacagtattgcaaaatataatttccctgatagtcctgctgagtatttagattatattattgcaagtaaagaccatgcgaacccatcatatatagagaataaggtgttacagccgaaatctccacaatggactgttacatcgtggttccaaaaatatacgtataatgattactctgatcattatccagtagaggcgactatttctatgaagtagtctaga

SEQ ID NO: 52 - DNA, 30 bp, Artificial sequence
  Forward primer for pCIP-CbPLC
catatgatgaataagaaaaaaatattaaaa

SEQ ID NO: 53 - DNA, 30 bp, Artificial sequence
  Reverse primer for pCIP-CbPLC
tctagattatttattatttatatagaatgt

SEQ ID NO: 54 - DNA, 1212 bp, Artificial sequence
  Insert for pCIP-CbPLC
catatgatgaataagaaaaaaatattaaaatttatttgtagtgcagtattatcattcacattattttcaggctataaaagttatgcatgggatggaaaacctgatggtacaggaactcatgcagtaatagttacacaagctgtagaaatgttaaaaaatgatgttataagtacatcacctttaagtgtaaaagaaaattttaaaattttagaatctaatttaaaaaaattacaacatggttctacttatccagattatgatccaaaggcatatgcattatatcaagatcatttttgggatcctgatacagataataattttactaaagatagtaaatggtatctatcatatgcaattagtcaaacaggagaatctcaacttagaaagttatttgcattagctaaagatgagtggaaaaaaggaaattatgaacaagcaacatggcttttaggacaaggtttacattactttggagatttcaatactccttatcatccatctaatgttacagctgtagatagtacaggtcatgtgaaatttgaaacttatgttgaagaaagaaaagattcatataaattaaattcagcaggaactaatagtgtaaaggaattttatctaactacattacaaaatactaatcttgataattggataacagagtattctagaggttgggctaaaaaagctaagaatatgtattatgctcatgctactatgaatcataattggaaagattgggaaatagcagctaatgaaactatgcataatgttcaaataggtagtgctggaataatatacagattccttaatgaagtatcaggaacaataaatacaactgaaaattctaaaataaatgaaataatgatagtaataaaaactgcagatgaagataaagcaggtacagatcattctattcatttcggaattgaagcaaaagatggaaagaagtatgaatggactcttgataatccaggtaatgattttgaaaaaaatcaagaagatagttatagaattaatttaaaagataataaattaacacttcaagatatagctaaaacatggataagaaaagaaagaggcgctggagttcaagatgattggaaacctgaatatgtaaaagtaattataaattcagatgttaagtatcaagctaatattaatgagtggtttggagataacaaaacattctatataaataataaataatctaga

SEQ ID NO: 55 - DNA, 30 bp, Artificial sequence; identical to SEQ ID NO: 46
  Forward primer for pCIP-CbC3

SEQ ID NO: 56 - DNA, 29 bp, Artificial sequence
  Reverse primer for pCIP-CbC3
tcctctagagaagtgacttagtcacttca

SEQ ID NO: 57 - DNA, 769 bp, Artificial sequence
  Insert for pCIP-CbC3
ggcatatgaaaggtttaagaaaatcaattttatgtttagttttgtcagcaggagtaatagctccagtaacatctgggatgattcaaagtcctcaaaaatgttatgcttattccattaatcaaaaggcttattcaaatacttaccaggagtttactaatattgatcaagcaaaagcttggggtaatgctcagtataaaaagtatggactaagcaaatcagaaaaagaagctatagtatcatatactaaaagcgctagtgaaataaatggaaagctaagacaaaataagggagttatcaatggatttccttcaaatttaataaaacaagttgaacttttagataaatcttttaataaaatgaagacccctgaaaatattatgttatttagaggcgacgaccctgcttatttaggaacagaatttcaaaacactcttcttaattcaaatggtacaattaataaaacggcttttgaaaaggctaaagctaagtttttaaataaagatagacttgaatatggatatattagtacttcattaatgaatgtttctcaatttgcaggaagaccaattattacaaaatttaaagtagcaaaaggctcaaaggcaggatatattgaccctattagtgcttttgcaggacaacttgaaatgttgcttcctagacatagtacttatcatatagacgatatgagattgtcttctgatggtaaacaaataataattacagcaacaatgatgggcacagctatcaatcctaaataatctagagg

SEQ ID NO: 58 - DNA, 6283 bp, Artificial sequence
  pCIP-CpA
aagcttctagattattttatattataagttgaatttcctgaaatccactcgtttatatctttgtccactacaacttttccatttgctattatctttatgttttctggcttataagcatctgagaatgctgtatattttctttttctaatccacatattttgtatatcatcaatttttagattttcatcttttaatttgaaagtataagtgtctttacttccagtcataaaatcatttcctgggttgtccatttcccattcttgagtttttccatcctttgttttgattccaaaatacatgtagtcatctgttccagcatctttctcaccactagttgatatgtaagctactagttcttttacattctttccaactgatggatcattaccctctgatacatcgtgtaagaatctataaatatatcccgctgttcctttttgagagttagctaaagttacctttgctgcataatcccaatcatcccaactatgactcatgctagcatgactatagtatattgattttcctgttttagcaaaacctcttgcatattcttttgaccatgcattaaaatctttgttttttaagatatcagtataaaaagcctcattagttttgcaacctgctgtgtttattttatactgttcttttctttcctctgcaaaagtctcaaacttaacatgtcctgcgctatcaacggcagtaacattagcaggatgatatggagtatctatatctccaaaatagtgcatagcctctccaagatagaatgtagcttgtttatagtttcctctttgccattcatatctagctaatgctgaaaattttcttatttgtgattcccctgtgtcaggtatagaataagctaaataccaactattatcctttgagaaattattatctgtatcaggatcccagaaatgatcttgatatagatcatatgcattcttatcataatctggataagtagaacctaattgaagctcatgcatgttctcttttaaaatctctaagttttttcttacactttctggttcatttttggacagatcattttctaagattgaaaccccttgagttacaatcatagcatgagttcctgttccatcaatctttccatcccaagcgtagactttagttgatgccccagcccataggctagttgctagcgcggcacaaataagcgccttacaaatctttcttttcatatgactccctcctaattcaagtttatttaagattcaatcttcaaataatgaaaatatattttaaatttttctttttaacataagataacctcctttctattttgaatttagttataatattatgtattaatattatataaatattatacagtatatgacaaagaaaatctctatataaatacaaaaaatatatttttatcaataaaagtaatttaatgtaaaattagattgtattttctgaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccc

SEQ ID NO: 59 - DNA, 5884 bp, Artificial sequence
  pCIP-CpB2
aagcttctagactatgcacaatatccttcaccaaatactctaattgatgcaggtgtaaagttttgtattcttattctatatttatagaaagattgtcctttttctatatataatcttatttgtctagcagaatcagggttttgaccatacaccatttgagaagctttaacatcatctccccataaccaatgatctacaacactatattctacttttaaatctcttgaacctgaagttgcatattgtctaaagtttaataatatattgtctgcataatatgtaaatctagtagaatatgtttttggatcaactcccccaattgattgtgataattcatcatcccatgtacaaacttgagttctaaatgatggtgttaaaatatattttccctcattctcaatttttgttctttccatgtcagatactattaattccccatctttagatgatacagtaaatttcatatcataaattgcaccattcataaattcagtttttacattaaatatttctttattatcaatcttttgtgaattaagtatttcaaaagattttagttgttggttaggatcatatttaaactcttctaacatctctttatacttttcatcactattcactctttcatcttctgatacgtttacaatcgtattaatatcgtagtttttaaaagcattaagataattctccattacctttctataagcgtcgatttcctttgcacttgctttcattggacttattgctccaacaataagaaaatatgaaaacataaaaattacagtaaactttgaaataatttttttcatatgactccctcctaattcaagtttatttaagattcaatcttcaaataatgaaaatatattttaaatttttctttttaacataagataacctcctttctattttgaatttagttataatattatgtattaatattatataaatattatacagtatatgacaaagaaaatctctatataaatacaaaaaatatatttttatcaataaaagtaatttaatgtaaaattagattgtattttctgaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccc

SEQ ID NO: 60 - DNA, 6106 bp, Artificial sequence
  pCIP-BcSMase
aagcttctagactacttcatagaaatagtcgcctctactggataatgatcagagtaatcattatacgtatatttttggaaccacgatgtaacagtccattgtggagatttcggctgtaacaccttattctctatatatgatgggttcgcatggtctttacttgcaataatataatctaaatactcagcaggactatcagggaaattatattttgcaatactgtttgtcgttgcgtcccaagtcgctgtatgtccagtataagatggtacagaagcgttcaatgttttaaacatagatgcatactctgaatcattcttgttctctgcattaattttatttacgttcatatcaccgccaattaacacatactcattatttggtatatttttatttttaataaagtcttgaatttcttttaattggtctgtacgtacagatgcaggtgaagtttttccgcacatactatcttccgcttgcaaatgtgtaccaatcacgtgaacgaagcgatcatttttcttaattttcgtgtatacaaatcctttattcgataaattatctggcccgcatccttttgcaaatacatattgaatcttttcagcgattggccatttgctcacaatcgcaacgccaccatcttcaggagttgaagatgaatagtttcctaacgttttatcccattcacttccactactacgacctaatactgctgtttgatttggatattctttcttcaaattccctaataaacgatctgaagcgctattatcaaacacttcatttaatataactacatcttgattctttatataatctgccgccccgattaaatcagcacgctcagtttgtccccagttcggatataagtttgttgatagcatatatacattatgcgtcatcacttttaatgtatcattttgatttgtagacacttctgcttgagctgacgttccgctatataaagctcctaaaccaacacctaagctaagtacaccttttagcaatttacatttcacgttccatacctccatcatatgactccctcctaattcaagtttatttaagattcaatcttcaaataatgaaaatatattttaaatttttctttttaacataagataacctcctttctattttgaatttagttataatattatgtattaatattatataaatattatacagtatatgacaaagaaaatctctatataaatacaaaaaatatatttttatcaataaaagtaatttaatgtaaaattagattgtattttctgaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccc

SEQ ID NO: 61 - DNA, 6289 bp, Artificial sequence
  pCIP-CbPLC
aagcttctagattatttattatttatatagaatgttttgttatctccaaaccactcattaatattagcttgatacttaacatctgaatttataattacttttacatattcaggtttccaatcatcttgaactccagcgcctctttcttttcttatccatgttttagctatatcttgaagtgttaatttattatcttttaaattaattctataactatcttcttgatttttttcaaaatcattacctggattatcaagagtccattcatacttctttccatcttttgcttcaattccgaaatgaatagaatgatctgtacctgctttatcttcatctgcagtttttattactatcattatttcatttattttagaattttcagttgtatttattgttcctgatacttcattaaggaatctgtatattattccagcactacctatttgaacattatgcatagtttcattagctgctatttcccaatctttccaattatgattcatagtagcatgagcataatacatattcttagcttttttagcccaacctctagaatactctgttatccaattatcaagattagtattttgtaatgtagttagataaaattcctttacactattagttcctgctgaatttaatttatatgaatcttttctttcttcaacataagtttcaaatttcacatgacctgtactatctacagctgtaacattagatggatgataaggagtattgaaatctccaaagtaatgtaaaccttgtcctaaaagccatgttgcttgttcataatttccttttttccactcatctttagctaatgcaaataactttctaagttgagattctcctgtttgactaattgcatatgatagataccatttactatctttagtaaaattattatctgtatcaggatcccaaaaatgatcttgatataatgcatatgcctttggatcataatctggataagtagaaccatgttgtaatttttttaaattagattctaaaattttaaaattttcttttacacttaaaggtgatgtacttataacatcattttttaacatttctacagcttgtgtaactattactgcatgagttcctgtaccatcaggttttccatcccatgcataacttttatagcctgaaaataatgtgaatgataatactgcactacaaataaattttaatatttttttcttattcatcatatgactccctcctaattcaagtttatttaagattcaatcttcaaataatgaaaatatattttaaatttttctttttaacataagataacctcctttctattttgaatttagttataatattatgtattaatattatataaatattatacagtatatgacaaagaaaatctctatataaatacaaaaaatatatttttatcaataaaagtaatttaatgtaaaattagattgtattttctgaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccc

SEQ ID NO: 62 - DNA, 5842 bp, Artificial sequence
  pCIP-CbC3
aagcttctagattatttaggattgatagctgtgcccatcattgttgctgtaattattatttgtttaccatcagaagacaatctcatatcgtctatatgataagtactatgtctaggaagcaacatttcaagttgtcctgcaaaagcactaatagggtcaatatatcctgcctttgagccttttgctactttaaattttgtaataattggtcttcctgcaaattgagaaacattcattaatgaagtactaatatatccatattcaagtctatctttatttaaaaacttagctttagccttttcaaaagccgttttattaattgtaccatttgaattaagaagagtgttttgaaattctgttcctaaataagcagggtcgtcgcctctaaataacataatattttcaggggtcttcattttattaaaagatttatctaaaagttcaacttgttttattaaatttgaaggaaatccattgataactcccttattttgtcttagctttccatttatttcactagcgcttttagtatatgatactatagcttctttttctgatttgcttagtccatactttttatactgagcattaccccaagcttttgcttgatcaatattagtaaactcctggtaagtatttgaataagccttttgattaatggaataagcataacatttttgaggactttgaatcatcccagatgttactggagctattactcctgctgacaaaactaaacataaaattgattttcttaaacctttcatatgactccctcctaattcaagtttatttaagattcaatcttcaaataatgaaaatatattttaaatttttctttttaacataagataacctcctttctattttgaatttagttataatattatgtattaatattatataaatattatacagtatatgacaaagaaaatctctatataaatacaaaaaatatatttttatcaataaaagtaatttaatgtaaaattagattgtattttctgaattcctgttataaaaaaaggatcaattttgaactctctcccaaagttgatcccttaacgatttagaaatccctttgagaatgtttatatacattcaaggtaaccagccaactaatgacaatgattcctgaaaaaagtaataacaaattactatacagataagttgactgatcaacttccataggtaacaacctttgatcaagtaagggtatggataataaaccacctacaattgcaatacctgttccctctgataaaaagctggtaaagttaagcaaactcattccagcaccagcttcctgctgtttcaagctacttgaaacaattgttgatataactgttttggtgaacgaaagcccacctaaaacaaatacgattataattgtcatgaaccatgatgttgtttctaaaagaaaggaagcagttaaaaagctaacagaaagaaatgtaactccgatgtttaacacgtataaaggacctcttctatcaacaagtatcccaccaatgtagccgaaaataatgacactcattgttccagggaaaataattacacttccgatttcggcagtacttagctggtgaacatctttcatcatataaggaaccatagagacaaaccctgctactgttccaaatataattcccccacaaagaactccaatcataaaaggtatatttttccctaatccgggatcaacaaaaggatctgttactttcctgatatgttttacaaatatcaggaatgacagcacgctaacgataagaaaagaaatgctatatgatgttgtaaacaacataaaaaatacaatgcctacagacattagtataattcctttgatatcaaaatgaccttttatccttacttctttctttaataatttcataagaaacggaacagtgataattgttatcataggaatgagtagaagataggaccaatgaatataatgggctatcattccaccaatcgctggaccgactccttctcccatggctactatcgatccaataagaccaaatgctttacccctattttcctttggaatatagcgcgcaactacaaccattacgagtgctggaaatgcagctgcaccagccccttgaataaaacgagccataataagtaaggaaaagaaagaatggccaacaaacccaattaccgacccgaaacaatttattataattccaaataggagtaaccttttgatgcctaattgatcagatagctttccatatacagctgttccaatggaaaaggttaacataaaggctgtgttcacccagtttgtactcgcaggtggtttattaaaatcatttgcaatatcaggtaatgagacgttcaaaaccatttcatttaatacgctaaaaaaagataaaatgcaaagccaaattaaaatttggttgtgtcgtaaattcgattgtgaataggatgtattcacatttcaccctccaataatgagggcagacgtagtttatagggttaatgatacgcttccctcttttaattgaaccctgttacattcattattcattacacttcataattaattcctcctaaacttgattaaaacattttaccacatataaactaagttttaaattcagtatttcatcacttatacaacaatatggcccgtttgttgaactactctttaataaaataatttttccgttcccaattccacattgcaataatagaaaatccatcttcatcggctttttcgtcatcatctgtatgaatcaaatcgccttcttctgtgtcatcaaggtttaattttttatgtatttcttttaacaaaccaccataggagattaaccttttacggtgtaaaccttcctccaaatcagacaaacgtttcaaattcttttcttcatcatcggtcataaaatccgtatcctttacaggatattttgcagtttcgtcaattgccgattgtatatccgatttatatttatttttcggtcgaatcatttgaacttttacatttggatcatagtctaatttcattgcctttttccaaaattgaatccattgtttttgattcacgtagttttctgtattcttaaaataagttggttccacacataccaatacatgcatgtgctgattataagaattatctttattatttattgtcacttccgttgcacgcataaaaccaacaagatttttattaatttttttatattgcatcattcggcgaaatccttgagccatatctgacaaactcttatttaattcttcgccatcataaacatttttaactgttaatgtgagaaacaaccaacgaactgttggcttttgtttaataacttcagcaacaaccttttgtgactgaatgccatgtttcattgctctcctccagttgcacattggacaaagcctggatttacaaaaccacactcgatacaactttctttcgcctgtttcacgattttgtttatactctaatatttcagcacaatcttttactctttcagcctttttaaattcaagaatatgcagaagttcaaagtaatcaacattagcgattttcttttctctccatggtctcacttttccactttttgtcttgtccactaaaacccttgatttttcatctgaataaatgctactattaggacacataatattaaaagaaacccccatctatttagttatttgtttggtcacttataactttaacagatggggtttttctgtgcaaccaattttaagggttttccaatactttaaaacacatacataccaacacttcaacgcacctttcagcaactaaaataaaaatgacgttatttctatatgtatcaagataagaaagaacaagttcaaaaccatcaaaaaaagacaccttttcaggtgctttttttattttataaactcattccctgatctcgacttcgttctttttttacctctcggttatgagttagttcaaattcgttctttttaggttctaaatcgtgtttttcttggaattgtgctgttttatcctttaccttgtctacaaaccccttaaaaacgtttttaaaggcttttaagcgtctgtacgttccttaaggaattattccttagtgctttctaggttaatgtcatgataataatggtttcttagacgtcaggtggcacttttcggggaaatgtccgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggaggtttttcgaaggttctctgagctaccaactctttgaaccgaggtaactggcttgcaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccc